(19) United States
(12) Patent Application Publication
(10) Pub. No.: US 2024/0010739 A1
(43) Pub. Date: Jan. 11, 2024

(54) ANTIBODIES CONJUGATED OR FUSED TO THE RECEPTOR-BINDING DOMAIN OF THE SARS-COV-2 SPIKE PROTEIN AND USES THEREOF FOR VACCINE PURPOSES

(71) Applicants: Institut National de la Santé et de la Recherche Médicale (INSERM), Paris (FR); Assistance Publique - Hopitaux de Paris (APHP), Paris (FR); Baylor Research Institute, DALLAS, TX (US); Universite Paris Est Creteil Val De Marne, Creteil (FR)

(72) Inventors: Yves LEVY, Creteil Cedex (FR); Gérard ZURAWSKI, Dallas, TX (US); Mireille CENTLIVRE, Creteil Cedex (FR); Sandra ZURAWSKI, Dallas, TX (US); Véronique GODOT, Creteil Cedex (FR)

(73) Assignees: Institut National de la Santé et de la Recherche Médicale (INSERM), Paris (FR); Assistance Publique - Hopitaux de Paris (APHP), Paris (FR); Baylor Research Institute, Dallas, TX (US); Universite Paris Est Creteil Val De Marne, Creteil (FR)

(21) Appl. No.: 18/036,237

(22) PCT Filed: Nov. 10, 2021
(86) PCT No.: PCT/EP2021/081303

§ 371 Date: May 10, 2023

(30) Foreign Application Priority Data
Nov. 12, 2020 (EP) 20306370.6
Jan. 26, 2021 (EP) 21305091.7

(51) Int. Cl. C07K 16/28; C12N 15/63

(52) U.S. Cl. CPC C07K 16/2875; C07K 16/2851; C12N 15/63; C07K 2317/24; C07K 2317/565; A61K 2039/505

(57) ABSTRACT

SARS-CoV-2 vaccines will be essential to reduce morbidity and mortality. The inventors produced an antibody that is directed against a surface antigen (i.e. CD40) of an antigen presenting cell (i.e. dendritic cell) wherein the heavy chain was conjugated to the receptor-binding domain of the Sars-Cov-2 spike protein for its use as vaccine. In particular, the inventors show that said vaccine induces circulating Ab-secreting hu-B cells, elicits S-specific IgG+ hu-B cells, elicits the expansion of central memory CD4+ hu-T cells and the emergence of effector memory CD4+ T cells, elicits the expansion of central memory CD8+ hu-T cells at and the emergence of effector memory CD8+ T cells at and finally induces Stem-cell like memory hu-CD8+ T cells. The present invention thus relates to antibodies that are directed against a surface antigen of an antigen presenting cell wherein the heavy chain and/or the light chain is conjugated or fused to the receptor-binding domain of the Sars-Cov-2 spike protein

### CROSS-REFERENCE TO RELATED APPLICATIONS

This application is a U.S. National Stage Application pursuant to 35 U.S.C. § 371 of International Patent Application PCT/EP2021/081303, filed on Nov. 10, 2021, and published as WO 2022/101302 on May 19, 2022, which claims priority to European Patent Application 21305091.7, filed on Jan. 26, 2021, and European Patent Application No. 20306370.6, filed on Nov. 12, 2020, all of which are incorporated herein by reference in their entireties for all purposes.

### FIELD OF THE INVENTION

The present invention is in the field of medicine, in particular virology and vaccinology.

### BACKGROUND OF THE INVENTION

The Severe Acute Respiratory Syndrome coronavirus 2 (SARS-CoV-2) which started in Wuhan, China, in December 2019 induced a threat to global health. In Mar. 11 2020, the WHO declared COVID-19 as a pandemic. The rapidity, rate of global spread and observed enhanced mortality raises public health, socio-economic and scientific challenges. As of yet, as it seems to spread very actively, it has infected more than 185 countries with more than 4,100,000 confirmed cases, and more than 280,000 deaths as of May 10 2020. SARS-CoV-2 can cause a respiratory syndrome that manifests a clinical pathology resembling mild upper respiratory tract disease (common cold-like symptoms) and occasionally severe lower respiratory tract illness and extra-pulmonary manifestations leading to multi-organ failure and death. This pandemic follows several highly pathogenic human coronaviruses infections including SARS-CoV in 2002 with a death rate of 10% and MERS-CoV in 2012 with a death rate of 36%. No treatment or vaccines are available. However, SARS-CoV-2 vaccines will be essential to reduce morbidity and mortality if the virus establishes itself in the population.

### SUMMARY OF THE INVENTION

The present invention is defined by the claims. In particular the present invention relates to antibodies that are directed against a surface antigen of an antigen presenting cell wherein the heavy chain and/or the light chain is conjugated or fused to the receptor-binding domain of the Sars-Cov-2 spike protein.

### DETAILED DESCRIPTION OF THE INVENTION

Definitions:

As used herein, the term “subject” or “subject in need thereof”, is intended for a human or non-human mammal. Typically the patient is affected or likely to be infected with SARS-Cov-2. As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions.

As used herein, the term “Severe Acute Respiratory Syndrome coronavirus 2” or “SARS-Cov-2” has its general meaning in the art and refers to the strain of coronavirus that causes coronavirus disease 2019 (COVID-19), a respiratory syndrome that manifests a clinical pathology resembling mild upper respiratory tract disease (common cold-like symptoms) and occasionally severe lower respiratory tract illness and extra-pulmonary manifestations leading to multi-organ failure and death. In particular, the term refers to the severe acute respiratory syndrome coronavirus 2 isolate 2019-nCoV_HKU-SZ-005b_2020 for which the complete genome is accessible under the NCBI access number MN975262.

As used herein, the term “Covid-19” refers to the respiratory disease induced by the Severe Acute Respiratory Syndrome coronavirus 2.

As used herein, the term “asymptomatic” refers to a subject who experiences no detectable symptoms for the coronavirus infection. As used herein, the term “symptomatic” refers to a subject who experiences detectable symptoms of coronavirus infection. Symptoms of coronavirus infection include: fatigue, anosmia, headache, cough, fever, difficulty to breathe.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “mutation” has its general meaning in the art and refers to a substitution, deletion or insertion. In particular, the term “substitution” means that a specific amino acid residue at a specific position is removed and another amino acid residue is inserted into the same position. Within the specification, the mutation are references according to the standard mutation nomenclature. In particular the term “mutation” encompasses “naturally-occurring mutations” and “non-naturally occurring mutations”.

As used herein, the term “naturally occurring mutation” refers to any mutation that can be found in the naturally occurring variants of the SARS-CoV-2 polypeptides and that typically include the B.1.1.7 lineage (a.k.a. 20I/501Y.V1 Variant of Concern (VOC) 202012/01), the B.1.351 lineage (a.k.a. 20H/501Y.V2) and the P.1 lineage (a.k.a. 20J/501Y.V3). Said mutation are well-known in the art and include those described in the following references that are incorporated by reference:
(1) Jie Hu et al. The D614G mutation of SARS-CoV-2 spike protein enhances viral infectivity and decreases neutralization sensitivity to individual convalescent sera. bioRxviv (2020).
(2) Korber B. et al. Spike mutation pipeline reveals the emergence of a more transmissible form of SARS-CoV-2. bioRxviv (2020). doi.org/10.1101/2020.04.29.069054.
(3) Lizhou Zhang et al. The D614G mutation in the SARS-CoV-2 spike protein reduces 51 shedding and increases infectivity. bioRxviv (2020). doi.org/10.1101/2020.06.12.148726.
(4) Junxian Ou et al. Emergence of RBD mutations in circulating SARS-CoV-2 strains enhancing the structural stability and human ACE2 receptor affinity of the spike protein. bioRxiv (2020). doi:10.1101/2020.03.15.991844v4
(5) Saha, P. et al. Mutations in Spike Protein of SARS-CoV-2 Modulate Receptor Binding, Membrane Fusion and Immunogenicity: An Insight into Viral Tropism and Pathogenesis of COVID-19. chemRxiv (2020). doi:10.26434/chemrxiv.12320567.v1
(6) Jian Shang, Yushun Wan, Chuming Luo, Gang Ye, Qibin Geng, Ashley Auerbach, Fang Li. Cell entry mechanisms of SARS-CoV-2. Proceedings of the National Academy of Sciences May 2020, 117 (21) 11727-11734; DOI:
(7) Allison J. Greaney, Andrea N. Loes, Katharine H. D. Crawford, Tyler N. Starr, Keara D. Malone, Helen Y. Chu, Jesse D. Bloom, bioRxiv 2020.12.31.425021; doi: https://doi.org/10.1101/2020.12.31.425021
(8) Nicholas G. Davies, Rosanna C. Barnard, Christopher I. Jarvis, Adam J. Kucharski, James Munday, Carl A. B. Pearson, Timothy W. Russell, Damien C. Tully, Sam Abbott, Amy Gimma, William Waites, Kerry L M Wong, Kevin van Zandvoort, CMMID COVID-19 Working Group, Rosalind M. Eggo, Sebastian Funk, Mark Jit, Katherine E. Atkins, W. John Edmunds. Estimated transmissibility and severity of novel SARS-CoV-2 Variant of Concern 202012/01 in England. medRxiv 2020.12.24.20248822; doi: https://doi.org/10.1101/2020.12.24.20248822
(9) Houriiyah Tegally, Eduan Wilkinson, Marta Giovanetti, et al. Emergence and rapid spread of a new severe acute respiratory syndrome-related coronavirus 2 (SARS-CoV-2) lineage with multiple spike mutations in South Africa. medRxiv 2020.12.21.20248640; doi: https://doi.org/10.1101/2020.12.21.20248640
(10) Kim J S, Jang J H, Kim J M, Chung Y S, Yoo C K, Han M G. Genome-Wide Identification and Characterization of Point Mutations in the SARS-CoV-2 Genome. Osong Public Health Res Perspect. 2020;11(3):101-111. doi:10.24171/j.phrp.2020.11.3.05
(11) Nilgiriwala K, Mandal A, Patel G, Mestry T, Vaswani S, Shaikh A, Sriraman K, Parikh S, Udupa S, Chatterjee N, Shastri J, Mistry N. Genome Sequences of Five SARS-CoV-2 Variants from Mumbai, India, Obtained by Nanopore Sequencing. Microbiol Resour Announc. 2021 Apr. 15;10(15):e00231-21
(12) Wenjuan Zhang, Brian D Davis, Stephanie S Chen, Jorge M Sincuir Martinez, Jasmine T Plummer, Eric Vail. Emergence of a Novel SARS-CoV-2 Variant in Southern California. JAMA. 2021 Apr. 6;325(13):1324-1326

For instance, the mutation N501Y is a non-synonymous mutation within the S-protein's receptor binding domain (RBD) shared by the three SARS-CoV-2 lineages B.1.1.7, P.1 (a.k.a. 20J/501Y.V3) and 501Y.V2 first identified in southeastern England, Brasil/Japan and South Africa respectively. It is one of the key contact residues within the RBD and has been identified as increasing binding affinity to human and murine ACE2. The E484K mutation within the S-protein' s receptor binding domain (RBD), present in the novel lineages 501Y.S2 and B.1.1.28 from South Africa and Brazil respectively, affects a residue within the RBD that has been shown to be important for binding of many neutralizing antibodies. The E484Q mutation within the S-protein's receptor binding domain (RBD), present in the novel lineages B.1.617 and B.1.429 from India and Denmark respectively, affects also the same residue within the RBD. Studies suggested that the L452R mutation in the lineages B.1.617, B1.427 and B1.429 may stabilize the interaction between the spike protein and its human ACE2 receptor and thereby increase infectivity of the virus. Accordingly, this mutation affects antibody recognition and enable SARS-CoV-2 immune escape. Virus bearing this mutation has been shown to escape recognition by antibodies in peoples' convalescent sera and may thus alter the effectiveness of vaccines (see e.g. Allison J. Greaney, Andrea N. Loes, Katharine H. D. Crawford, Tyler N. Starr, Keara D. Malone, Helen Y. Chu, Jesse D. Bloom, bioRxiv 2020.12.31.425021). Several other mutations have been discovered. The mutations K417N, K417T, V367F, N354D, W436R or V483A, T478K of the 51 protein have been shown to bind with higher affinity to ACE2. V483A and G4765 mutations have previously been reported to be related to human receptor-binding affinity in MERS and SARS-CoV research. R408I on the other hand potentially reduce the ACE2 binding affinity. According to the present invention the main naturally occurring mutations thus include, the K417N mutation in SEQ ID NO:1 wherein the amino acid residue (K) at position 417 in SEQ ID NO:1 is substituted by the amino acid residue (N), the K417T mutation in SEQ ID NO:1 wherein the amino acid residue (K) at position 417 in SEQ ID NO:1 is substituted by the amino acid residue (T), the E484K mutation in SEQ ID NO:1 wherein the amino acid residue (E) at position 484 in SEQ ID NO:1 is substituted by the amino acid residue (K), the E484Q mutation in SEQ ID NO:1 wherein the amino acid residue (E) at position 484 is substituted by the amino acid residue (Q), the L452R mutation in SEQ ID NO:1 wherein the amino acid residue (L) at position 452 in SEQ ID NO:1 is substituted by the amino acid residue (R), the T478K mutation in SEQ ID NO:1 wherein the amino acid residue (T) at position 478 is substituted by the amino acid residue (K) and the N501Y mutation in SEQ ID NO:1 wherein the amino acid residue (N) at position 501 in SEQ ID NO:1 is substituted by the amino acid residue (Y).

As used herein, the term “non-naturally occurring mutation” refers to any mutation that are genetically inserted in the polypeptides of the present invention. In particular, said mutations are inserted to ease the production of the polypeptide. For instance, said mutations include the mutation C538S in SEQ ID NO:1 wherein the amino acid residue (C) at position 538 in SEQ ID NO:1 is substituted by the amino acid residue (S). Said mutations are particularly suitable for avoiding the creation of disulphide bonds within the polypeptide of the present invention. As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

As used herein, the term “promoter/regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame. As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

As used herein, the term “expression system” means a host cell and compatible vector under suitable conditions, e.g., for the expression of a protein coded for by foreign DNA carried by the vector and introduced to the host cell.

As used herein, the term “spike protein” or “protein S” refers to the SARS-Cov-2 spike glycoprotein that binds its cellular receptor (i.e. ACE2), and mediates membrane fusion and virus entry. Each monomer of trimeric S protein is about 180 kDa, and contains two subunits, S1 and S2, mediating attachment and membrane fusion, respectively. In particular, Spike protein S1 attaches the virion to the cell membrane by interacting with host receptor (i.e. human ACE2 receptor) via its “receptor-binding domain” also named “RBD.” Spike protein S2 mediates fusion of the virion and cellular membranes by acting as a class I viral fusion protein. Under the current model, the protein has at least three conformational states: pre-fusion native state, pre-hairpin intermediate state, and post-fusion hairpin state. During viral and target cell membrane fusion, the coiled coil regions (heptad repeats) assume a trimer-of-hairpins structure, positioning the fusion peptide in close proximity to the C-terminal region of the ectodomain. The formation of this structure appears to drive apposition and subsequent fusion of viral and target cell membranes. Spike protein S2′ acts as a viral fusion peptide which is unmasked following S2 cleavage occurring upon virus endocytosis. Typically, the spike protein has the amino acid sequence as set forth in SEQ ID NO:1.

>sp|P0DTC2|SPIKE_SARS2 Spike glycoprotein
OS = Severe acute respiratory syndrome
coronavirus 2 OX = 2697049 GN = S PE = 1
SV = 1.
SEQ ID NO: 1
The RBD is underlined in the sequence.
MFVFLVLLPLVSSQCVNLTTRTQLPPAYTNSFTRGVYYPDK
VFRSSVLHSTQDLFLPFFSNVTWFHAIHVSGTNGTKRFDN
PVLPENDGVYFASTEKSNIIRGWIFGTTLDSKTQSLLIVN
NATNVVIKVCEFQFCNDPFLGVYYHKNNKSWMESEFRVYS
SANNCTFEYVSQPFLMDLEGKQGNEKNLREFVFKNIDGYF
KIYSKHTPINLVRDLPQGFSALEPLVDLPIGINITRFQTL
LALHRSYLTPGDSSSGWTAGAAAYYVGYLQPRTELLKYNE
NGTITDAVDCALDPLSETKCTLKSFTVEKGIYQTSNERVQ
PTESIVRFPNITNLCPFGEVENATRFASVYAWNRKRISNC
VADYSVLYNSASFSTFKCYGVSPTKLNDLCFTNVYADSFV
IRGDEVRQIAPGQTGKIADYNYKLPDDFTGCVIAWNSNNL
DSKVGGNYNYLYRLERKSNLKPFERDISTEIYQAGSTPCN
GVEGFNCYFPLQSYGFQPTNGVGYQPYRVVVLSFELLHAP
ATVCGPKKSTNLVKNKCVNFNENGLTGTGVLTESNKKELP
FQQFGRDIADTTDAVRDPQTLEILDITPCSFGGVSVITPG
TNTSNQVAVLYQDVNCTEVPVAIHADQLTPTWRVYSTGSN
VFQTRAGCLIGAEHVNNSYECDIPIGAGICASYQTQTNSP
RRARSVASQSIIAYTMSLGAENSVAYSNNSIAIPTNFTIS
VTTEILPVSMTKTSVDCTMYICGDSTECSNLLLQYGSFCT
QLNRALTGIAVEQDKNTQEVFAQVKQIYKTPPIKDFGGEN
FSQILPDPSKPSKRSFIEDLLENKVTLADAGFIKQYGDCL
GDIAARDLICAQKENGLTVLPPLLTDEMIAQYTSALLAGT
ITSGWTFGAGAALQIPFAMQMAYRENGIGVTQNVLYENQK
LIANQFNSAIGKIQDSLSSTASALGKLQDVVNQNAQALNT
LVKQLSSNFGAISSVLNDILSRLDKVEAEVQIDRLITGRL
QSLQTYVTQQLIRAAEIRASANLAATKMSECVLGQSKRVD
FCGKGYHLMSFPQSAPHGVVFLHVTYVPAQEKNFTTAPAI
CHDGKAHFPREGVFVSNGTHWFVTQRNFYEPQIITTDNTF
VSGNCDVVIGIVNNTVYDPLQPELDSFKEELDKYFKNHTS
PDVDLGDISGINASVVNIQKEIDRLNEVAKNLNESLIDLQ
ELGKYEQYIKWPWYIWLGFIAGLIAIVMVTIMLCCMTSCC
SCLKGCCSCGSCCKFDEDDSEPVLKGVKLHYT

As used herein the term “RBD polypeptide” refers to the polypeptide that consists of the amino acid sequence having at least 90% of identity with the amino acid sequence that ranges from the amino acid residue at position 319 to the amino acid residue at position 541 in SEQ ID NO:1.

In some embodiments, the RBD polypeptide consists of the amino acid that ranges from the amino acid residue at position 319 to the amino acid residue at position 541 in SEQ ID NO:1.

In some embodiments, the RBD polypeptide consists of the amino acid that ranges from the amino acid residue at position 319 to the amino acid residue at position 541 in SEQ ID NO:1 and that comprises one or more non-naturally occurring mutation(s). In some embodiments, the RBD polypeptide consists of the amino acid that ranges from the amino acid residue at position 319 to the amino acid residue at position 541 in SEQ ID NO:1 and that comprises a non-naturally mutation at position 538. In some embodiments, the RBD polypeptide consists of the amino acid that ranges from the amino acid residue at position 319 to the amino acid residue at position 541 in SEQ ID NO:1 and that comprises the C538S mutation.

In some embodiments, the RBD polypeptide consists of the amino acid that ranges from the amino acid residue at position 319 to the amino acid residue at position 541 in SEQ ID NO:1 and that comprises one or more naturally occurring mutations (s). In some embodiments, the RBD polypeptide consists of the amino acid that ranges from the amino acid residue at position 319 to the amino acid residue at position 541 in SEQ ID NO:1 and that comprises one or more naturally occurring mutation(s) at position 417, 452, 478, 484 or 501. In some embodiments, the RBD polypeptide consists of the amino acid that ranges from the amino acid residue at position 319 to the amino acid residue at position 541 in SEQ ID NO:1 and that comprises one or more naturally occurring mutation(s) at position selected from the group consisting of K417N, K417T, E484K and N501Y mutations. In some embodiments, the RBD polypeptide consists of the amino acid that ranges from the amino acid residue at position 319 to the amino acid residue at position 541 in SEQ ID NO:1 and that comprises one or more naturally occurring mutation(s) at position selected from the group consisting of K417N, K417T, L452R, T478K, E484Q, E484K and N501Y mutations.

In some embodiments, the RBD polypeptide consists of the amino acid that ranges from the amino acid residue at position 319 to the amino acid residue at position 541 in SEQ ID NO:1 and that comprises the N501Y naturally occurring mutation and the C538S non naturally occurring mutation.

In some embodiments, the RBD polypeptide consists of the amino acid that ranges from the amino acid residue at position 319 to the amino acid residue at position 541 in SEQ ID NO:1 and that comprises the K417T, E484K, N501Y naturally-occurring mutations and the non-naturally occurring mutation C538S mutation.

In some embodiments, the RBD polypeptide consists of the amino acid that ranges from the amino acid residue at position 319 to the amino acid residue at position 541 in SEQ ID NO:1 and that comprises the K417N, E484K, N501Y naturally occurring mutations and the non-naturally occurring C538S mutation.

In some embodiments, the RBD polypeptide consists of the amino acid that ranges from the amino acid residue at position 319 to the amino acid residue at position 541 in SEQ ID NO:1 and that comprises the K417T, E484Q, N501Y naturally-occurring mutations and the non-naturally occurring mutation C538S mutation.

In some embodiments, the RBD polypeptide consists of the amino acid that ranges from the amino acid residue at position 319 to the amino acid residue at position 541 in SEQ ID NO:1 and that comprises the K417N, E484Q, N501Y naturally occurring mutations and the non-naturally occurring C538S mutation.

In some embodiments, the RBD polypeptide consists of the amino acid that ranges from the amino acid residue at position 319 to the amino acid residue at position 541 in SEQ ID NO:1 and that comprises the K417N, T478K, E484Q, L452R and N501Y naturally occurring mutations and the non-naturally occurring C538S mutation.

In some embodiments, the RBD polypeptide consists of the amino acid that ranges from the amino acid residue at position 319 to the amino acid residue at position 541 in SEQ ID NO:1 and that comprises the K417N, T478K, E484K, L452R and N501Y naturally occurring mutations and the non-naturally occurring C538S mutation.

As used herein, the term “conjugate” or interchangeably “conjugated polypeptide” is intended to indicate a composite or chimeric molecule formed by the covalent attachment of one or more polypeptides. The term “covalent attachment” or “conjugation” means that the polypeptide and the non-peptide moiety are either directly covalently joined to one another, or else are indirectly covalently joined to one another through an intervening moiety or moieties, such as a bridge, spacer, or linkage moiety or moieties. A particular conjugate is a fusion protein.

As used herein, the term “fusion protein” indicates a protein created through the attaching of two or more polypeptides which originated from separate proteins. In particular fusion proteins can be created by recombinant DNA technology and are typically used in biological research or therapeutics. Fusion proteins can also be created through chemical covalent conjugation with or without a linker between the polypeptides portion of the fusion proteins. In the fusion protein the two or more polypeptide are fused directly or via a linker.

As used herein, the term “directly” means that the first amino acid at the N-terminal end of a first polypeptide is fused to the last amino acid at the C-terminal end of a second polypeptide. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

As used herein, the term “linker” has its general meaning in the art and refers to an amino acid sequence of a length sufficient to ensure that the proteins form proper secondary and tertiary structures. In some embodiments, the linker is a peptidic linker which comprises at least one, but less than 30 amino acids e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation (i.e., a conformation allowing a proper signal transducing activity through the IL-signalling 15Rbeta/gamma signalling pathway). The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non hypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-(H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk—see the section entitled «How to identify the CDRs by looking at a sequence» within the Antibodies pages.

As used herein, the term “immunoglobulin domain” refers to a globular region of an antibody chain (such as e.g. a chain of a heavy chain antibody or a light chain), or to a polypeptide that essentially consists of such a globular region.

As used herein, the term “Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, of an agonist molecule, e.g., CD40 Ligand, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

As used herein, the term “immune response” refers to a reaction of the immune system to an antigen in the body of a host, which includes generation of an antigen-specific antibody and/or cellular cytotoxic response. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by the transgene. The level of the immune response can be measured by methods known in the art (e.g., by measuring antibody titre).

As used herein the term “APCs” or “Antigen Presenting Cells” denotes cells that are capable of activating T-cells, and include, but are not limited to, certain macrophages, B cells and dendritic cells.

As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells).

As used herein, the term “CD40” has its general meaning in the art and refers to human CD40 polypeptide receptor. In some embodiments, CD40 is the isoform of the human canonical sequence as reported by UniProtKB-P25942 (also referred as human TNR5).

As used herein, the term “CD40L” has its general meaning in the art and refers to human CD40L polypeptide, for example, as reported by UniProtKB-P25942, including its CD40-binding domain of SEQ ID NO:47. CD40L may be expressed as a soluble polypeptide and is the natural ligand of CD40 receptor.

> CD40L binding domain
SEQ ID NO: 47
MQKGDQNPQIAAHVISEASSKTTSVLQWAEKGYYTMSNNL
VTLENGKQLTVKRQGLYYIYAQVTFCSNREASSQAPFIAS
LCLKSPGRFERILLRAANTHSSAKPCGQQSIHLGGVFELQ
PGASVFVNVTDPSQVSHGTGFTSFGLLKL

As used herein, the term “CD40 agonist antibody” is intended to refer to an antibody that increases CD40 mediated signaling activity in the absence of CD40L in a cell-based assay, such as the B cell proliferation assay. In particular, the CD40 agonist antibody (i) it induces the proliferation of B cell, as measured in vitro by flow cytometric analysis, or by analysis of replicative dilution of CFSE-labeled cells; and/or (ii) induces the secretion of cytokines, such as IL-6, IL-12, or IL-15, as measured in vitro with a dendritic cell activation assay.

As used herein, the term “Langerin” has its general meaning in the art and refers to human C-type lectin domain family 4 member K polypeptide. In some embodiments, Langerin is the isoform of the human canonical sequence as reported by UniProtKB-Q9UJ71 (also referred as human CD207).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccination” or “vaccinating” means, but is not limited to, a process to elicit an immune response in a subject against a particular antigen.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein the term “antigen” refers to a molecule capable of being specifically bound by an antibody or by a T cell receptor (TCR) if processed and presented by MHC molecules. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “adjuvant” refers to a compound a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dentritic cells (DCs).

As used herein, the expression “therapeutically effective amount” is meant a sufficient amount of the active ingredient of the present invention to induce an immune response at a reasonable benefit/risk ratio applicable to the medical treatment.

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. , 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA.

Antibodies of the Present Invention:

The first object of the present invention relates to an antibody that is directed against a surface antigen of an antigen presenting cell wherein the heavy chain and/or the light chain is conjugated or fused to the RBD polypeptide.

In some embodiments, the heavy chain of the antibody is conjugated or fused to the RBD polypeptide.

In some embodiments, the light chain of the antibody is conjugated or fused to the RBD polypeptide.

In some embodiments, both the heavy and light chains of the antibody are conjugated or fused to the RBD polypeptide.

In some embodiment, the heavy chain is fused or conjugated to the RBD polypeptide that consists of the amino acid that ranges from the amino acid residue at position 319 to the amino acid residue at position 541 in SEQ ID NO:1 and that comprises the C538S non naturally occurring mutation and the light chain is conjugated or fused to the RBD polypeptide consists of the amino acid that ranges from the amino acid residue at position 319 to the amino acid residue at position 541 in SEQ ID NO:1 and that comprises the K417N, E484K, N501Y naturally occurring mutation and the C538S non-naturally occurring mutation.

In some embodiment, the light chain is fused or conjugated to the RBD polypeptide that consists of the amino acid that ranges from the amino acid residue at position 319 to the amino acid residue at position 541 in SEQ ID NO:1 and that comprises the C538S non-naturally occurring mutation and the heavy chain is conjugated or fused to the RBD polypeptide consists of the amino acid that ranges from the amino acid residue at position 319 to the amino acid residue at position 541 in SEQ ID NO:1 and that comprises the K417N, E484K, N501Y naturally-occurring mutation and the C538S non-naturally occurring mutation.

In some embodiment, the heavy chain is fused or conjugated to the RBD polypeptide that consists of the amino acid that ranges from the amino acid residue at position 319 to the amino acid residue at position 541 in SEQ ID NO:1 and that comprises the C538S non naturally occurring mutation and the light chain is conjugated or fused to the RBD polypeptide consists of the amino acid that ranges from the amino acid residue at position 319 to the amino acid residue at position 541 in SEQ ID NO:1 and that comprises the K417N, E484Q, N501Y naturally occurring mutation and the C538S non-naturally occurring mutation.

In some embodiment, the light chain is fused or conjugated to the RBD polypeptide that consists of the amino acid that ranges from the amino acid residue at position 319 to the amino acid residue at position 541 in SEQ ID NO:1 and that comprises the C538S non-naturally occurring mutation and the heavy chain is conjugated or fused to the RBD polypeptide consists of the amino acid that ranges from the amino acid residue at position 319 to the amino acid residue at position 541 in SEQ ID NO:1 and that comprises the K417N, E484Q, N501Y naturally-occurring mutation and the C538S non-naturally occurring mutation.

In some embodiment, the heavy chain is fused or conjugated to the RBD polypeptide that consists of the amino acid that ranges from the amino acid residue at position 319 to the amino acid residue at position 541 in SEQ ID NO:1 and that comprises the C538S non naturally occurring mutation and the light chain is conjugated or fused to the RBD polypeptide consists of the amino acid that ranges from the amino acid residue at position 319 to the amino acid residue at position 541 in SEQ ID NO:1 and that comprises the K417N, L452R, T478K, E484Q, N501Y naturally occurring mutation and the C538S non-naturally occurring mutation.

In some embodiment, the light chain is fused or conjugated to the RBD polypeptide that consists of the amino acid that ranges from the amino acid residue at position 319 to the amino acid residue at position 541 in SEQ ID NO:1 and that comprises the C538S non naturally occurring mutation and the heavy chain is conjugated or fused to the RBD polypeptide consists of the amino acid that ranges from the amino acid residue at position 319 to the amino acid residue at position 541 in SEQ ID NO:1 and that comprises the K417N, L452R, T478K, E484Q, N501Y naturally occurring mutation and the C538S non-naturally occurring mutation.

In some embodiment, the heavy chain is fused or conjugated to the RBD polypeptide that consists of the amino acid that ranges from the amino acid residue at position 319 to the amino acid residue at position 541 in SEQ ID NO:1 and that comprises the C538S non naturally occurring mutation and the light chain is conjugated or fused to the RBD polypeptide consists of the amino acid that ranges from the amino acid residue at position 319 to the amino acid residue at position 541 in SEQ ID NO:1 and that comprises the K417N, L452R, T478K, E484K, N501Y naturally occurring mutation and the C538S non-naturally occurring mutation.

In some embodiment, the light chain is fused or conjugated to the RBD polypeptide that consists of the amino acid that ranges from the amino acid residue at position 319 to the amino acid residue at position 541 in SEQ ID NO:1 and that comprises the C538S non naturally occurring mutation and the heavy chain is conjugated or fused to the RBD polypeptide consists of the amino acid that ranges from the amino acid residue at position 319 to the amino acid residue at position 541 in SEQ ID NO:1 and that comprises the K417N, L452R, T478K, E484K, N501Y naturally occurring mutation and the C538S non-naturally occurring mutation.

In some embodiments, the antibody is an IgG antibody, preferably of an IgG1 or IgG4 antibody, or even more preferably of an IgG4 antibody.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the antibody is a human antibody. In some embodiments, human antibodies can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). In another embodiment, human anti-PD-1 antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

In some embodiments, the antibody is specific for a cell surface marker of a professional APC. The antibody may be specific for a cell surface marker of another professional APC, such as a B cell or a macrophage.

In some embodiments, the antibody is selected from an antibody that specifically binds to DC immunoreceptor (DCIR), MHC class I, MHC class II, CD1, CD2, CD3, CD4, CD8, CD1 1b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fey receptor, LOX-1, and ASPGR.

In some embodiments, the antibody is specific for CD40.

In some embodiments, the anti-CD40 antibody derives from the 12E12 antibody and comprises:
a heavy chain comprising the complementarity determining regions CDR1H, CDR2H and CDR3H, the CDR1H having the amino acid sequence GFTFSDYYMY (SEQ ID NO:2), the CDR2H having the amino acid sequence YINSGGGSTYYPDTVKG (SEQ ID NO:3), and the CDR3H having the amino acid sequence RGLPFHAMDY (SEQ ID NO:4),
and a light chain comprising the complementarity determining regions CDR1L, CDR2L and CDR3L, the CDR1L having the amino acid sequence SASQGISNYLN (SEQ ID NO:5) the CDR2L having the amino acid sequence YTSILHS (SEQ ID NO:6) and the CDR3L having the amino acid sequence QQFNKLPPT (SEQ ID NO:7).

In some embodiments, the anti-CD40 antibody derives from the 11B6 antibody and comprises:
a heavy chain comprising the complementarity determining regions CDR1H, CDR2H and CDR3H, the CDR1H having the amino acid sequence GYSFTGYYMH (SEQ ID NO:8), the CDR2H having the amino acid sequence RINPYNGATSYNQNFKD (SEQ ID NO:9), and the CDR3H having the amino acid sequence EDYVY (SEQ ID NO:10), and
a light chain comprising the complementarity determining regions CDR1L, CDR2L and CDR3L, the CDR1L having the amino acid sequence RSSQSLVHSNGNTYLH (SEQ ID NO:11) the CDR2L having the amino acid sequence KVSNRFS (SEQ ID NO:12) and the CDR3L having the amino acid sequence SQSTHVPWT (SEQ ID NO:13).

In some embodiments, the anti-CD40 antibody derives from the 12B4 antibody and comprises:
a heavy chain comprising the complementarity determining regions CDR1H, CDR2H and CDR3H, the CDR1H having the amino acid sequence GYTFTDYVLH (SEQ ID NO:14), the CDR2H having the amino acid sequence YINPYNDGTKYNEKFKG (SEQ ID NO:15), and the CDR3H having the amino acid sequence GYPAYSGYAMDY (SEQ ID NO:16), and
a light chain comprising the complementarity determining regions CDR1L, CDR2L and CDR3L, the CDR1L having the amino acid sequence RASQDISNYLN (SEQ ID NO:17) the CDR2L having the amino acid sequence YTSRLHS (SEQ ID NO:18) and the CDR3L having the amino acid sequence HHGNTLPWT (SEQ ID NO:19).

In some embodiments, the anti-CD40 antibody is selected from the group consisting of selected mAb1, mAb2, mAb3, mAb4, mAb5 and mAb6 as described in Table A.

TABLE A
CD40 antibodies

| | | |
|---|---|---|
| mAb1 [11B6 VH/VkV2] | SEQ ID NO: 20 | SEQ ID NO: 21 |
| mAb2 [11B6 VHV3/VkV2] | SEQ ID NO: 22 | SEQ ID NO: 21 |
| mAb3 [12B4] | SEQ ID NO: 23 | SEQ ID NO: 24 |
| mAb4 [24A3] | SEQ ID NO: 25 | SEQ ID NO: 26 |
| mAb5 [CP870, 893] | SEQ ID NO: 27 | SEQ ID NO: 28 |
| mAb 6 [12E12] | SEQ ID NO: 29 | SEQ ID NO: 30 |

(Amino acid sequence of variable heavy
chain region (VH) (v2) of Humanized 11B6)
SEQ ID NO: 20
EVQLVQSGAEVKKPGASVKISCKASGYSFTGYYMHWVKQA
HGQGLEWIGRINPYNGATSYNQNFKDRATLTVDKSTSTAY
MELSSLRSEDTAVYYCAREDYVYWGQGTTVTVSSAS
(Amino acid sequence of variable light
chain (VL) Vk (v2) of humanized 11B6 VL)
SEQ ID NO: 21
DVVMTQSPLSLPVTLGQPASISCRSSQSLVHSNGNTYLHWY
QQRPGQSPRLLIYKVSNRFSGVPDRESGSGSGTDFTLKIS
RVEAEDVGVYFCSQSTHVPWTFGGGTK
(Amino acid sequence of variable heavy
chain region VH (v3) of humanized 11B6)
SEQ ID NO: 22
EVQLVQSGAEVKKPGASVKVSCKASGYSFTGYYMHWVRQA
PGQGLEWIGRINPYNGATSYNQNFKDRVTLTVDKSTSTAY
MELSSLRSEDTAVYYCAREDYVYWGQGTTVTVSSAS
(VH amino acid sequence of mAb3 (12B4))
SEQ ID NO: 23
EVQLQQSGPELVKPGASVKMSCKASGYTFTDYVLHWVKQK
PGQGLEWIGYINPYNDGTKYNEKFKGKATLTSDKSSSTAY
MELSSLTSEDSAVYYCARGYPAYSGYAMDYWGQGTSVTVS
SAS
(VL amino acid sequence of mAb3 (12B4))
SEQ ID NO: 24
DIQMTQTTSSLSASLGDRVTISCRASQDISNYLNWYQQKP
DGTVKLLIYYTSRLHSGVPSRESGSGSGTDYSLTISNLEQ
EDIATYFCHHGNTLPWTFGGGTK
(VH amino acid sequence of mAb4 (24A3 HC))
SEQ ID NO: 25
DVQLQESGPDLVKPSQSLSLTCTVTGYSITSDYSWHWIRQ
FPGNKLEWMGYIYYSGSTNYNPSLKSRISITRDTSKNQFF
LQLNSVTTEDSATYFCARFYYGYSFFDYWGQGTTLTVSSA
S
(VL amino acid sequence of mAb4 (24A3 KC))
SEQ ID NO: 26
QIVLTQSPAFMSASPGEKVTMTCSASSSVSYMHWYQQKSG
TSPKRWIYDTSKLASGVPARFSGSGSGTSYSLTISSMEAE
DAATYYCQQWSSNPLTFGAGTK
(VH amino acid sequence of mAb5)
SEQ ID NO: 27
QVQLVQSGAEVKKPGASVKVSCKASGYTFTGYYMHWVRQA
PGQGLEWMGWINPDSGGTNYAQKFQGRVTMTRDTSISTAY
MELNRLRSDDTAVYYCARDQPLGYCTNGVCSYFDYWGQGT
LVTVSSAS
(VL amino acid sequence of mAb5)
SEQ ID NO: 28
DIQMTQSPSSVSASVGDRVTITCRASQGIYSWLAWYQQKP
GKAPNLLIYTASTLQSGVPSRESGSGSGTDFTLTISSLQP
EDFATYYCQQANIFPLTFGGGTK
(VH amino acid sequence of mAb6
(12E12 H3 Humanized HC))
SEQ ID NO: 29
EVQLVESGGGLVQPGGSLKLSCATSGFTFSDYYMYWVRQA
PGKGLEWVAYINSGGGSTYYPDTVKGRFTISRDNAKNTLY
LQMNSLRAEDTAVYYCARRGLPFHAMDYWGQGTLVTVSSA
S
(VL amino acid sequence of mAb6
(Humanized K2 12E12))
SEQ ID NO: 30
DIQMTQSPSSLSASVGDRVTITCSASQGISNYLNWYQQKP
GKAVKLLIYYTSILHSGVPSRESGSGSGTDYTLTISSLQP
EDFATYYCQQFNKLPPTFGGGTK

In some embodiments, the anti-CD40 antibody is a CD40 agonist antibody. CD40 agonist antibodies are described in WO2010/009346, WO2010/104747 and WO2010/104749. Other anti-CD40 agonist antibodies in development include CP-870,893 that is a fully human IgG2 CD40 agonist antibody developed by Pfizer. It binds CD40 with a KD of 3.48×10−10 M, but does not block binding of CD40L (see e.g., U.S. Pat. No. 7,338,660) and SGN-40 that is a humanized IgG1 antibody developed by Seattle Genetics from mouse antibody clone S2C6, which was generated using a human bladder carcinoma cell line as the immunogen. It binds to CD40 with a KD of 1.0×10−9 M and works through enhancing the interaction between CD40 and CD40L, thus exhibiting a partial agonist effect (Francisco J A, et al., Cancer Res, 60: 3225-31, 2000). Even more particularly, the CD40 agonist antibody is selected from the group consisting of selected mAb1, mAb2, mAb3, mAb4, mAb5 and mAb6 as described in Table A.

In some embodiments, the heavy chain or the light chain of the CD40 agonist antibody (i.e. the chain that is not conjugated or fused to the RBD polypeptides) is conjugated or fused to a CD40 binding domain of CD40L.

In some embodiments, the CD40 binding domain of CD40L is fused to the C-terminus of a light or heavy chain of said CD40 agonist antibody, optionally via a linker, preferably the FlexV1 linker as described herein after.

In some embodiments, the antibody of the present invention consists of a CD40 agonist antibody wherein the heavy chain of the antibody is fused or conjugated to the RBD polypeptide and the light chain is conjugated or fused to the CD40 binding domain of CD40L (SEQ ID NO:47).

In some embodiments, the antibody is specific for Langerin. In some embodiments, the antibody derives from the antibody 15B10 having ATCC Accession No. PTA-9852. In some embodiments, the antibody derives from the antibody 2G3 having ATCC Accession No. PTA-9853. In some embodiments, the antibody derives from the antibody 91E7, 37C1, or 4C7 as described in WO2011032161.

In some embodiments, the anti-Langerin antibody comprises a heavy chain comprising the complementarity determining regions CDR1H, CDR2H and CDR3H of the 15B10 antibody and a light chain comprising the complementarity determining regions CDR1L, CDR2L and CDR3L of the 15B10 antibody.

In some embodiments, the anti-Langerin antibody comprises a heavy chain comprising the complementarity determining regions CDR1H, CDR2H and CDR3H of the 2G3 antibody and a light chain comprising the complementarity determining regions CDR1L, CDR2L and CDR3L of the 2G3 antibody.

In some embodiments, the anti-Langerin antibody comprises a heavy chain comprising the complementarity determining regions CDR1H, CDR2H and CDR3H of the 4C7 antibody and a light chain comprising the complementarity determining regions CDR1L, CDR2L and CDR3L of the 4C7 antibody.

In some embodiments, the antibody is selected from the group consisting of selected mAb7, mAb8, mAb9, mAb10, mAb11 and mAb12 as described in Table B.

| | | |
|---|---|---|
| mAb7 [15B10] | SEQ ID NO: 31 | SEQ ID NO: 32 |
| mAb8 [2G3] | SEQ ID NO: 33 | SEQ ID NO: 34 |
| mAb9 [4C7] | SEQ ID NO: 35 | SEQ ID NO: 36 |

(Amino acid sequence of variable
heavy chain region (VH) of 15B10)
SEQ ID NO: 31
SVKMSCKASGYTFTDYVISWVKQRTGQGLEWIGDIYPGSG
YSFYNENFKGKATLTADKSSTTAYMQLSSLTSEDSAVYFC
A
(Amino acid sequence of variable
light chain (VL) 15B10)
SEQ ID NO: 32
ASISCRSSQSLVHSNGNTYLHWYLQKPGQSPKLLIYKVSN
RFSGVPDRESGSGSGTNFTLKISRVEAEDLGLYFCS
(Amino acid sequence of variable
heavy chain region (VH) of 2G3)
SEQ ID NO: 33
SSVKMSCKASGYTFTDYVISWVKQRTGQGLEWIGDIYPGS
GYSFYNENFKGKATLTADKSSTTAYMQLSSLTSEDSAVYF
CA
(Amino acid sequence of variable
light chain (VL) 2G3)
SEQ ID NO: 34
VTLTCRSSTGAVTTSNYANWVQEKPDHLFTGLIGGTNNRV
SGVPARFSGSLIGDKAALTITGAQTEDEAIYFCA
(Amino acid sequence of the heavy
chain of 4C7)
SEQ ID NO: 35
QVQLQQSGAELVRPGASVTLSCKASGYTFIDHDMHWVQQT
PVYGLEWIGAIDPETGDTGYNQKFKGKAILTADKSSRTAY
MELRSLTSEDSAVYYCTIPFYYSNYSPFAYWGQGALVTVS
AAKTTAPSVYPLAPVCGGTTGSSVTLGCLVKGYFPEPVTL
TWNSGSLSSGVHTFPALLQSGLYTLSSSVTVTSNTWPSQT
ITCNVAHPASSTKVDKKIEPRVPITQNPCPPLKECPPCAD
LLGGPSVFIFPPKIKDVLMISLSPMVTCVVVDVSEDDPDA
QISWFVNNVEVHTAQTQTHREDYNSTLRVVSALPIQHQDW
MSGKEFKCKVNNRALPSPIEKTISKPRGPVRAPQVYVLPP
PAEEMTKKEFSLTCMITGFLPAEIAVDWTSNGRTEQNYKN
TATVLDSDGSYFMYSKLRVQKSTWERGSLFACSVVHEGLH
NHLTTKTISRSLGKAS
(Amino acid sequence of light chain
of 4C7)
SEQ ID NO: 36
QIVLSQSPAILSASPGEKVTMTCRASSSVSYMHWYQRKPG
SSPKPWIYATSNLASGVPARFSGSGSGTSYSLTISRVEAE
DAATYYCQQWSSNPLTFGAGTKLELKRADAAPTVSIFPPS
SEQLTSGGASVVCFLNNFYPKDINVKWKIDGSERQNGVLN
SWTDQDSKDSTYSMSSTLTLTKDEYERHNSYTCEATHKTS
TSPIVKSFNRNEC

The antibodies of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, the antibodies of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

The heavy chain and/or the light chain of the antibody is conjugated or fused to the RBD polypeptide via its c-terminus. In some embodiments, the heavy chain and/or the light chain of the antibody is fused to the N-terminus of the RBD polypeptide.

In some embodiments, the heavy chain and/or the light chain of the antibody is conjugated to the RBD polypeptide by using chemical coupling. Several methods are known in the art for the attachment or conjugation of an antibody to its conjugate moiety. Examples of linker types that have been used to conjugate a moiety to an antibody include, but are not limited to, hydrazones, thioethers, esters, disulfides and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). Techniques for conjugating polypeptides and in particular, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies ′84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58; see also, e.g., PCT publication WO 89/12624.) Typically, the peptide is covalently attached to lysine or cysteine residues on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res.16, 4769-4778). Junutula et al. (Nat Biotechnol. 2008; 26:925-32) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q- tags) or an endogenous glutamine that are made reactive by polypeptide engineering (e.g., via amino acid deletion, insertion, substitution, or mutation on the polypeptide). Then a transglutaminase can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In some embodiments, the heavy chain and/or the light chain of the antibody is conjugated to the RBD polypeptide by a dockerin domain or multiple domains to permit non-covalent coupling to cohesin fusion proteins as described in US20160031988A1 and US20120039916A1.

In some embodiments, the heavy chain and/or the light chain of the antibody is fused to the RBD polypeptide to form a fusion protein. In some embodiments, the RBD polypeptide is fused either directly or via a linker to the heavy chain or to the light chain. As used herein, the term “directly” means that the first amino acid at the N-terminal end of the RBD polypeptide is fused to the last amino acid at the C-terminal end of the heavy or of the light chain. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

In some embodiments, the linker is selected from the group consisting of FlexV1, f1, f2, f3, or f4 as described below.

(flexV1, SEQ ID NO: 37)
QTPTNTISVTPTNNSTPTNNSNPKPNP
(f1, SEQ ID NO: 38)
SSVSPTTSVHPTPTSVPPTPTKSSP
(f2, SEQ ID NO: 39)
PTSTPADSSTITPTATPTATPTIKG
(f3, SEQ ID NO: 40)
TVTPTATATPSAIVTTITPTATTKP
(f4, SEQ ID NO: 41)
TNGSITVAATAPTVTPTVNATPSAA

In some embodiments, the antibody comprises i) a heavy chain that is fused to the RBD polypeptide to form the fusion protein as set forth in SEQ ID NO:42 and ii) a light chain as set forth in SEQ ID NO:43.

(hAnti-CD40VH3-LV-hIgG4H-C-
AS-ViralSARS-CoV-2-Spike-RBDC221S)
SEQ ID NO: 42
EVQLVESGGGLVQPGGSLKLSCATSGFTFSDYYMYWVRQA
PGKGLEWVAYINSGGGSTYYPDTVKGRFTISRDNAKNTLY
LQMNSLRAEDTAVYYCARRGLPFHAMDYWGQGTLVTVSSA
STKGPSVEPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFEGGPSVF
LFPPKPKDTLMISRTPEVTCVVVDVSQEDPEVQFNWYVDG
VEVHNAKTKPREEQENSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLGKASRVQPTESIVRFPNITNLCPFGEVENATRFASV
YAWNRKRISNCVADYSVLYNSASFSTFKCYGVSPTKLNDL
CFTNVYADSFVIRGDEVRQIAPGQTGKIADYNYKLPDDFT
GCVIAWNSNNLDSKVGGNYNYLYRLERKSNLKPFERDIST
EIYQAGSTPCNGVEGENCYFPLQSYGFQPTNGVGYQPYRV
VVLSFELLHAPATVCGPKKSTNLVKNKSVNF
(hAnti-CD40VK2-LV-hIgGK-C)
SEQ ID NO:43
DIQMTQSPSSLSASVGDRVTITCSASQGISNYLNWYQQKP
GKAVKLLIYYTSILHSGVPSRESGSGSGTDYTLTISSLQP
EDFATYYCQQFNKLPPTFGGGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGEC

In some embodiments, the antibody comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:44.

[hAnti-CD40VK2-LV-hIgGK-C-ViralSARS-
CoV-2-Spike-RBDC221S SA var]
SEQ ID NO: 44
DIQMTQSPSSLSASVGDRVTITCSASQGISNYLNWYQQKP
GKAVKLLIYYTSILHSGVPSRFSGSGSGTDYTLTISSLQP
EDFATYYCQQFNKLPPTFGGGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGECASRVQPTESIVRFPNITNLCPFGEVE
NATRFASVYAWNRKRISNCVADYSVLYNSASFSTFKCYGV
SPTKLNDLCFTNVYADSFVIRGDEVRQIAPGQTGNIADYN
YKLPDDFTGCVIAWNSNNLDSKVGGNYNYLYRLFRKSNLK
PFERDISTEIYQAGSTPCNGVKGENCYFPLQSYGFQPTYG
VGYQPYRVVVLSFELLHAPATVCGPKKSTNLVKNKSVNF

In some embodiments, the antibody comprises i) a heavy chain that is fused to the RBD polypeptide to form the fusion protein as set forth in SEQ ID NO:45 and ii) a light chain that is fused to the RBD polypeptide to form the fusion protein as set forth in SEQ ID NO:46.

[hAnti-CD40VH3-LV-hIgGK-C-ViralSARS-
CoV-2-Spike-RBDC221S SA var]
SEQ ID NO: 45
EVQLVESGGGLVQPGGSLKLSCATSGFTFSDYYMYWVRQA
PGKGLEWVAYINSGGGSTYYPDTVKGRFTISRDNAKNTLY
LQMNSLRAEDTAVYYCARRGLPFHAMDYWGQGTLVTVSSA
STKGPSVFPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFEGGPSVF
LFPPKPKDTLMISRTPEVTCVVVDVSQEDPEVQFNWYVDG
VEVHNAKTKPREEQENSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVESCSVMHEALHNHYTQKSL
SLSLGKASRVQPTESIVRFPNITNLCPFGEVENATRFASV
YAWNRKRISNCVADYSVLYNSASFSTFKCYGVSPTKLNDL
CFTNVYADSFVIRGDEVRQIAPGQTGNIADYNYKLPDDFT
GCVIAWNSNNLDSKVGGNYNYLYRLERKSNLKPFERDIST
EIYQAGSTPCNGVKGFNCYFPLQSYGFQPTYGVGYQPYRV
VVLSFELLHAPATVCGPKKSTNLVKNKSVNE
[hAnti-CD40VK2-LV-hIgG4H-C-ViralSARS-
CoV-2-Spike-RBDC221S]
SEQ ID NO: 46
DIQMTQSPSSLSASVGDRVTITCSASQGISNYLNWYQQKP
GKAVKLLIYYTSILHSGVPSRESGSGSGTDYTLTISSLQP
EDFATYYCQQFNKLPPTFGGGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGECASRVQPTESIVRFPNITNLCPFGEVE
NATRFASVYAWNRKRISNCVADYSVLYNSASFSTFKCYGV
SPTKLNDLCFTNVYADSFVIRGDEVRQIAPGQTGKIADYN
YKLPDDFTGCVIAWNSNNLDSKVGGNYNYLYRLERKSNLK
PFERDISTEIYQAGSTPCNGVEGENCYFPLQSYGFQPTNG
VGYQPYRVVVLSFELLHAPATVCGPKKSTNLVKNKSVNF

In some embodiments, the antibody comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:48.

[hAnti-CD40VK2-LV-hIgGK-C-ViralSARS-
CoV-2-Spike-RBDC221S
(K417N, L452R, T478K, E484Q, N501Y)]
SEQ ID NO: 48
DIQMTQSPSSLSASVGDRVTITCSASQGISNYLNWYQQKP
GKAVKLLIYYTSILHSGVPSRFSGSGSGTDYTLTISSLQP
EDFATYYCQQFNKLPPTFGGGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGECASRVQPTESIVRFPNITNLCPFGEVE
NATRFASVYAWNRKRISNCVADYSVLYNSASFSTFKCYGV
SPTKLNDLCFTNVYADSFVIRGDEVRQIAPGQTGNIADYN
YKLPDDFTGCVIAWNSNNLDSKVGGNYNYRYRLERKSNLK
PFERDISTEIYQAGSKPCNGVQGENCYFPLQSYGFQPTYG
VGYQPYRVVVLSFELLHAPATVCGPKKSTNLVKNKSVNF

In some embodiments, the antibody comprises i) a heavy chain that is fused to the RBD polypeptide to form the fusion protein as set forth in SEQ ID NO:49 and ii) a light chain that is fused to the RBD polypeptide to form the fusion protein as set forth in SEQ ID NO:46.

[hAnti-CD40VH3-LV-hIgGK-C-ViralSARS-
CoV-2-Spike-RBDC221S
(K417N, L452R, T478K, E484Q, N501Y)]
SEQ ID NO: 49
EVQLVESGGGLVQPGGSLKLSCATSGFTFSDYYMYWVRQA
PGKGLEWVAYINSGGGSTYYPDTVKGRFTISRDNAKNTLY
LQMNSLRAEDTAVYYCARRGLPFHAMDYWGQGTLVTVSSA
STKGPSVFPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFEGGPSVF
LFPPKPKDTLMISRTPEVTCVVVDVSQEDPEVQFNWYVDG
VEVHNAKTKPREEQFNSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLGKASRVQPTESIVRFPNITNLCPFGEVENATRFASV
YAWNRKRISNCVADYSVLYNSASFSTFKCYGVSPTKLNDL
CFTNVYADSFVIRGDEVRQIAPGQTGNIADYNYKLPDDFT
GCVIAWNSNNLDSKVGGNYNYRYRLERKSNLKPFERDIST
EIYQAGSKPCNGVQGENCYFPLQSYGFQPTYGVGYQPYRV
VVLSFELLHAPATVCGPKKSTNLVKNKSVNF

In some embodiments, the antibody comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:50.

[hAnti-CD40VK2-LV-hIgGK-C-ViralSARS-
CoV-2-Spike-RBDC221S
(K417N, L452R, T478K, E484K, N501Y)]
SEQ ID NO: 50
DIQMTQSPSSLSASVGDRVTITCSASQGISNYLNWYQQKP
GKAVKLLIYYTSILHSGVPSRESGSGSGTDYTLTISSLQP
EDFATYYCQQFNKLPPTFGGGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGECASRVQPTESIVRFPNITNLCPFGEVE
NATRFASVYAWNRKRISNCVADYSVLYNSASFSTFKCYGV
SPTKLNDLCFTNVYADSFVIRGDEVRQIAPGQTGNIADYN
YKLPDDFTGCVIAWNSNNLDSKVGGNYNYRYRLERKSNLK
PFERDISTEIYQAGSKPCNGVKGENCYFPLQSYGFQPTYG
VGYQPYRVVVLSFELLHAPATVCGPKKSTNLVKNKSVNF

In some embodiments, the antibody comprises i) a heavy chain that is fused to the RBD polypeptide to form the fusion protein as set forth in SEQ ID NO:51 and ii) a light chain that is fused to the RBD polypeptide to form the fusion protein as set forth in SEQ ID NO:46.

[hAnti-CD40VH3-LV-hIgGK-C-ViralSARS-
CoV-2-Spike-RBDC221S
(K417N, L452R, T478K, E484K, N501Y)]
SEQ ID NO: 51
EVQLVESGGGLVQPGGSLKLSCATSGFTFSDYYMYWVRQA
PGKGLEWVAYINSGGGSTYYPDTVKGRFTISRDNAKNTLY
LQMNSLRAEDTAVYYCARRGLPFHAMDYWGQGTLVTVSSA
STKGPSVFPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFEGGPSVF
LFPPKPKDTLMISRTPEVTCVVVDVSQEDPEVQFNWYVDG
VEVHNAKTKPREEQFNSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLGKASRVQPTESIVRFPNITNLCPFGEVENATRFASV
YAWNRKRISNCVADYSVLYNSASFSTFKCYGVSPTKLNDL
CFTNVYADSFVIRGDEVRQIAPGQTGNIADYNYKLPDDFT
GCVIAWNSNNLDSKVGGNYNYRYRLERKSNLKPFERDIST
EIYQAGSKPCNGVKGENCYFPLQSYGFQPTYGVGYQPYRV
VVLSFELLHAPATVCGPKKSTNLVKNKSVNF

In some embodiments, the antibody comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:52.

[hAnti-CD40VK2-LV-hIgGK-C-ViralSARS-
CoV-2-Spike-RBDC221S
(K417N, E484Q, N501Y)]
SEQ ID NO: 52
DIQMTQSPSSLSASVGDRVTITCSASQGISNYLNWYQQKP
GKAVKLLIYYTSILHSGVPSRESGSGSGTDYTLTISSLQP
EDFATYYCQQFNKLPPTFGGGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGECASRVQPTESIVRFPNITNLCPFGEVE
NATRFASVYAWNRKRISNCVADYSVLYNSASFSTFKCYGV
SPTKLNDLCFTNVYADSFVIRGDEVRQIAPGQTGNIADYN
YKLPDDFTGCVIAWNSNNLDSKVGGNYNYLYRLERKSNLK
PFERDISTEIYQAGSTPCNGVQGFNCYFPLQSYGFQPTYG
VGYQPYRVVVLSFELLHAPATVCGPKKSTNLVKNKSVNF

In some embodiments, the antibody comprises i) a heavy chain that is fused to the RBD polypeptide to form the fusion protein as set forth in SEQ ID NO:53 and ii) a light chain that is fused to the RBD polypeptide to form the fusion protein as set forth in SEQ ID NO:46.

[hAnti-CD40VH3-LV-hIgGK-C-ViralSARS-
CoV-2-Spike-RBDC221S
(K417N, E484Q, N501Y)]
SEQ ID NO: 53
EVQLVESGGGLVQPGGSLKLSCATSGFTFSDYYMYWVRQA
PGKGLEWVAYINSGGGSTYYPDTVKGRFTISRDNAKNTLY
LQMNSLRAEDTAVYYCARRGLPFHAMDYWGQGTLVTVSSA
STKGPSVEPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFEGGPSVF
LFPPKPKDTLMISRTPEVTCVVVDVSQEDPEVQFNWYVDG
VEVHNAKTKPREEQFNSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLGKASRVQPTESIVRFPNITNLCPFGEVENATRFASV
YAWNRKRISNCVADYSVLYNSASFSTFKCYGVSPTKLNDL
CFTNVYADSFVIRGDEVRQIAPGQTGNIADYNYKLPDDFT
GCVIAWNSNNLDSKVGGNYNYLYRLERKSNLKPFERDIST
EIYQAGSTPCNGVQGENCYFPLQSYGFQPTYGVGYQPYRV
VVLSFELLHAPATVCGPKKSTNLVKNKSVNF

Nucleic Acids, Vectors and Host Cells of the Present Invention:

A further object of the invention relates to a nucleic acid that encodes for a heavy chain and/or the light chain of an antibody directed against a surface antigen of an antigen presenting cell that is fused to the RBD polypeptide.

Typically, said nucleic acid is a DNA or RNA molecule, which may be included in any suitable vector, such as a plasmid, cosmid, episome, artificial chromosome, phage or a viral vector.

So, a further object of the invention relates to a vector comprising a nucleic acid of the present invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a nucleic acid and/or a vector according to the invention.

The nucleic acids of the invention may be used to produce an antibody of the present invention in a suitable expression system. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E.coli, Kluyveromyces or Saccharomyces yeasts. Mammalian host cells include Chinese Hamster Ovary (CHO cells) including dhfr-CHO cells (described in Urlaub and Chasin, 1980) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing a polypeptide according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

The host cell as disclosed herein are thus particularly suitable for producing the antibody of the present invention. Indeed, when recombinant expression are introduced into mammalian host cells, the polypeptides are produced by culturing the host cells for a period of time sufficient for expression of the antibody in the host cells and, optionally, secretion of the antibody into the culture medium in which the host cells are grown. The antibodies can be recovered and purified for example from the culture medium after their secretion using standard protein purification methods.

Pharmaceutical and Vaccine Compositions:

The antibodies as described herein may be administered as part of one or more pharmaceutical compositions. Except insofar as any conventional carrier medium is incompatible with the antibodies of the present invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The antibodies as described herein are particularly suitable for preparing vaccine composition. Thus a further object of the present invention relates to a vaccine composition comprising an antibody of the present invention.

In some embodiments, the vaccine composition of the present invention comprises an adjuvant. In some embodiments, the adjuvant is alum. In some embodiments, the adjuvant is Incomplete Freund's adjuvant (IFA) or other oil based adjuvant that is present between 30-70%, preferably between 40-60%, more preferably between 45-55% proportion weight by weight (w/w). In some embodiments, the vaccine composition of the present invention comprises at least one Toll-Like Receptor (TLR) agonist which is selected from the group consisting of TLR1, TLR2, TLR3, TLR4, TLR5, TLR6, TLR7, and TLR8 agonists.

Therapeutic Methods:

The antibodies as well as the pharmaceutical or vaccine compositions as herein described are particularly suitable for inducing an immune response against SARS-Cov-2 and thus can be used for vaccine purposes.

Therefore, a further object of the present invention relates to a method for vaccinating a subject in need thereof against SARS-Cov 2 comprising administering a therapeutically effective amount of the antibody of the present invention.

In some embodiments, the antibodies as well as the pharmaceutical or vaccine compositions as herein described are particularly suitable for the treatment of Covid-19.

In some embodiments, the subject can be human or any other animal (e.g., birds and mammals) susceptible to coronavirus infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the subject can be symptomatic or asymptomatic.

Typically, the active ingredient of the present invention (i.e the antibodies and the pharmaceutical or vaccine compositions as herein described) is administered to the subject at a therapeutically effective amount. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The antibodies and the pharmaceutical or vaccine compositions as herein described may be administered to the subject by any route of administration and in particular by oral, nasal, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active agent which is being used.

In some embodiments, the antibodies as well as the pharmaceutical or vaccine compositions as herein described may be administered to the subject in combination with, for example, any known therapeutic agent or method for vaccinating against SARS-Cov-2 coronavirus. Non-limiting examples of such known therapeutics include but are not limited to anti-viral agents such as remdesivir, lopinavir, ritonavir, hydroxycholoroquine, and chloroquine. In some embodiments, the Antibodies and the pharmaceutical or vaccine compositions as herein described are administered in combination with an immune checkpoint inhibitor. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist. In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.570, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

The invention will be further illustrated by the following figures and examples. However, these examples and figures should not be interpreted in any way as limiting the scope of the present invention.

### FIGURES

FIG. 1. Immunogenicity of the aCD40-RBD vaccine given in homologous or heterologous prime/boost vaccination strategies. We purchased the NSG humanized mice from the Jackson Laboratories (USA). Five donors provided hematopoietic stem cells for human immune system reconstitution of the mice. Animals were kept at the Mondor Institute of Biomedical Research (U955 INSERM-Paris East Creteil University, Ile-de-France, France) following the recommendations of the French Ministry of Higher Education, Research and Innovation. The local ethics committee, ComEth Anses/ENVA/UPEC, approved the protocol, permit number 25329-2020051119073072 v4.

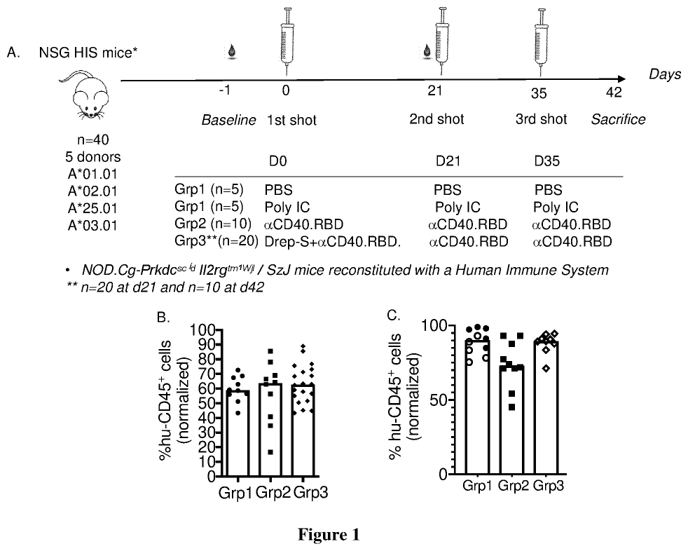

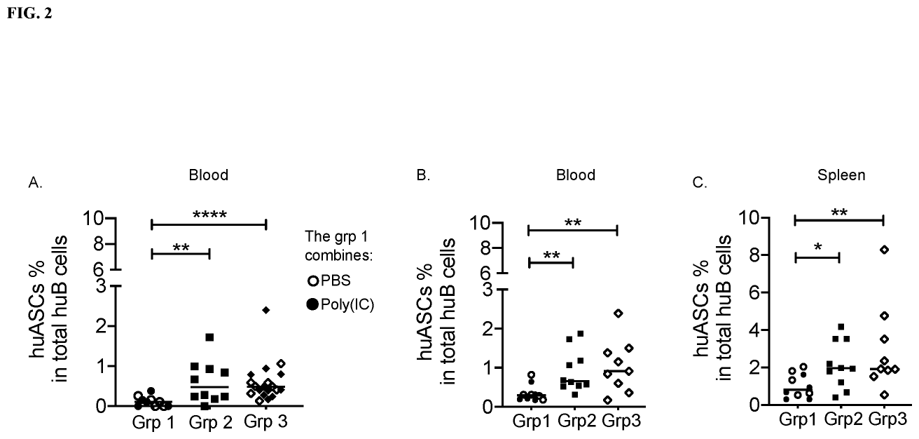

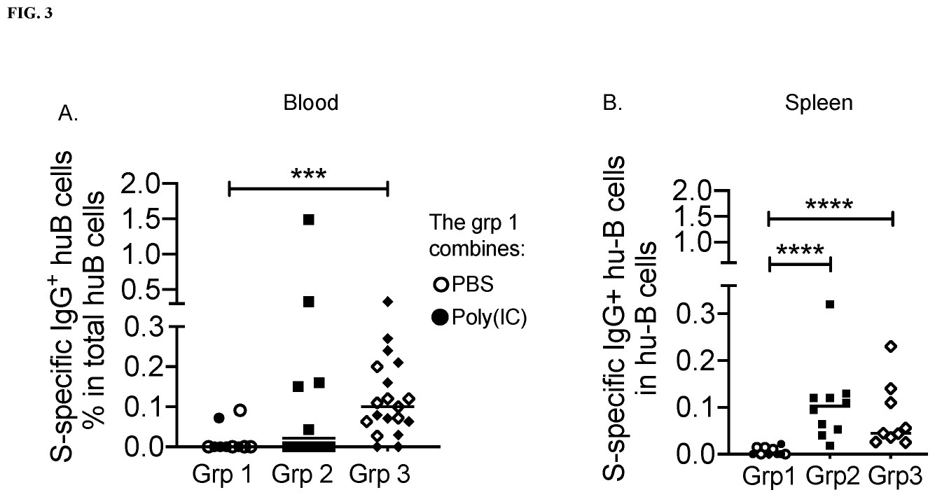

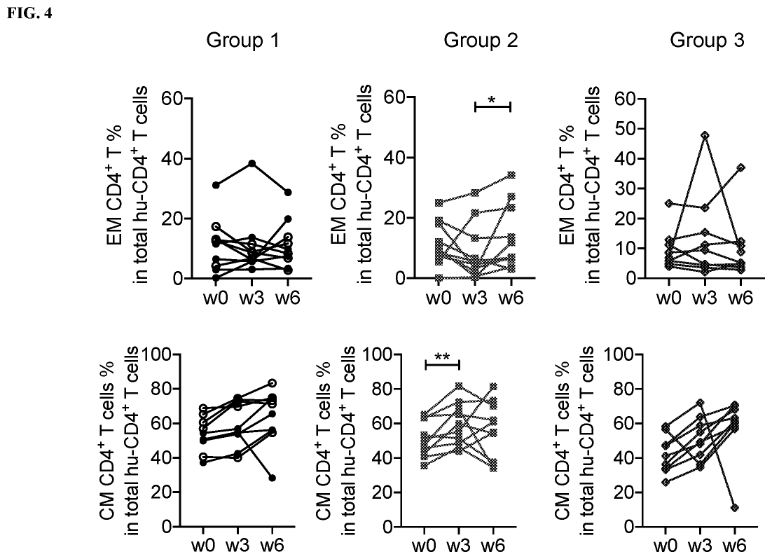

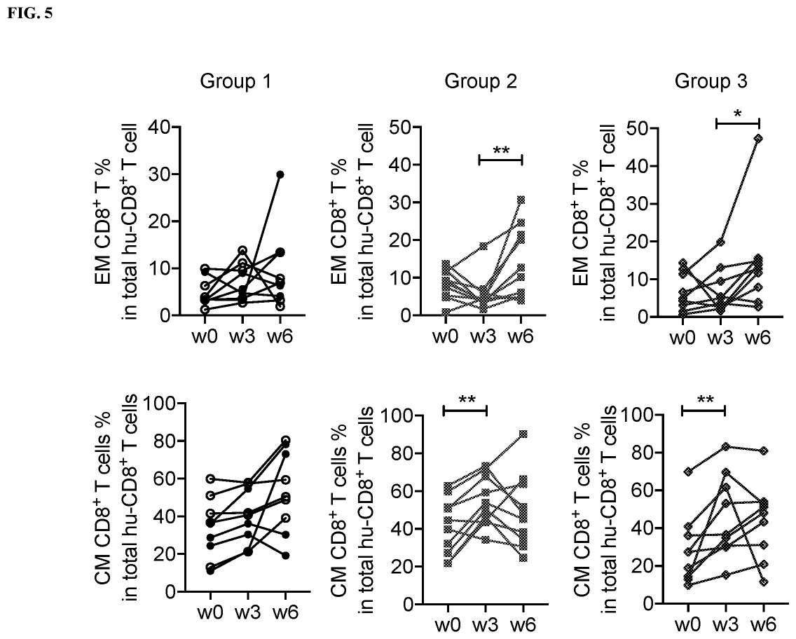

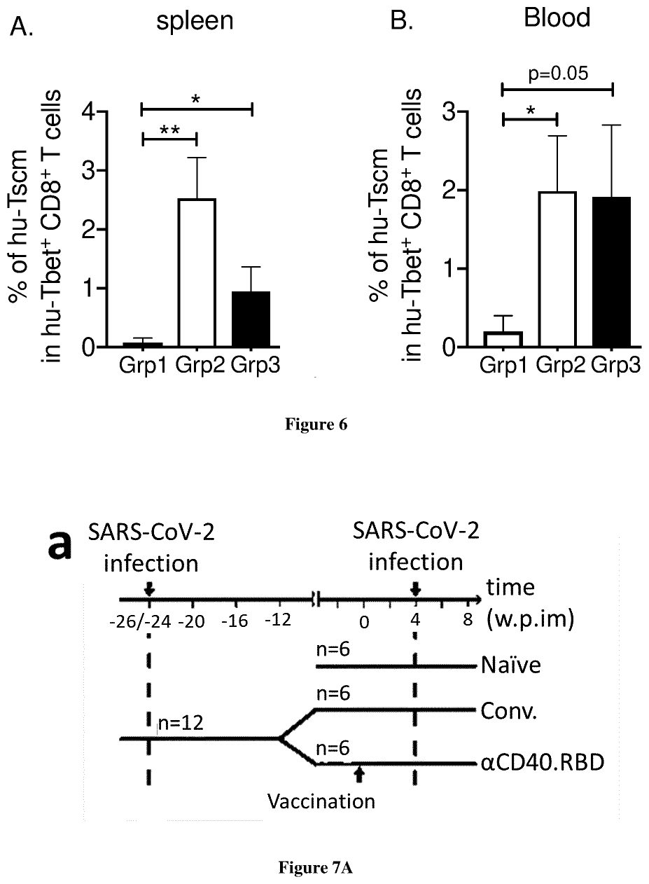

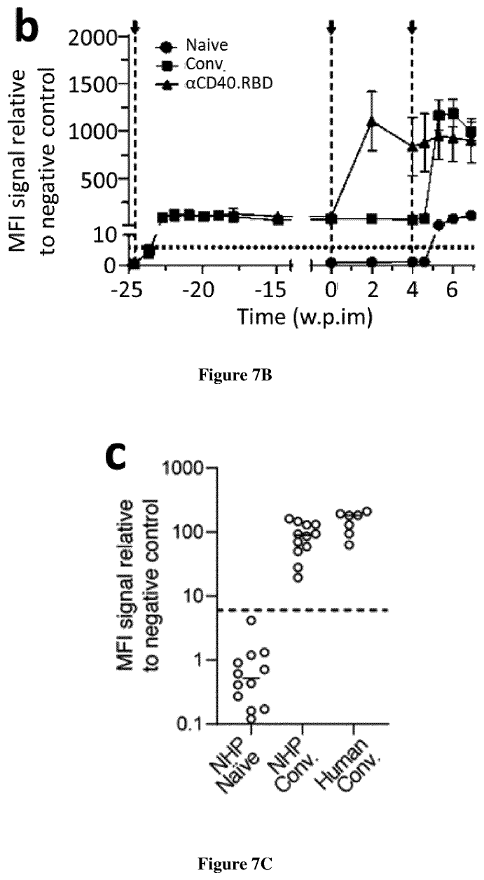

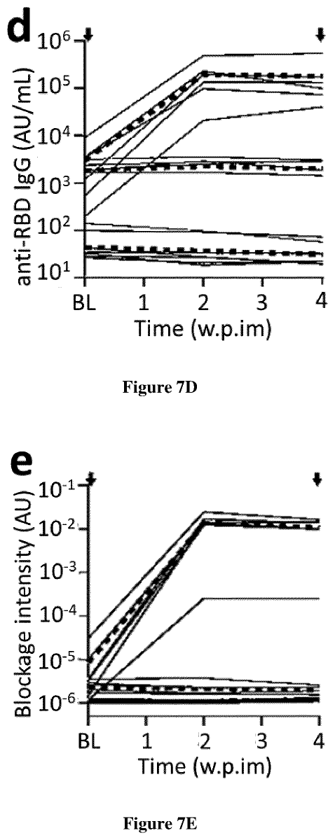

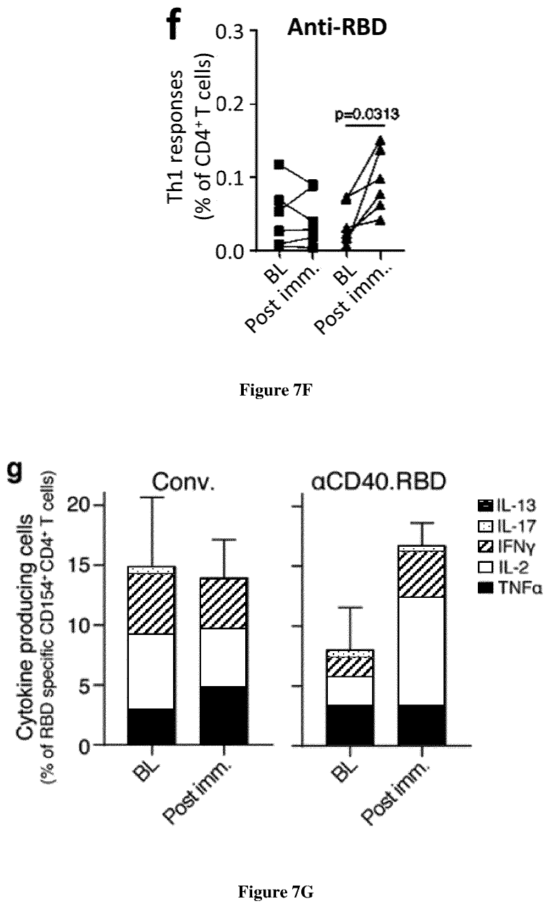

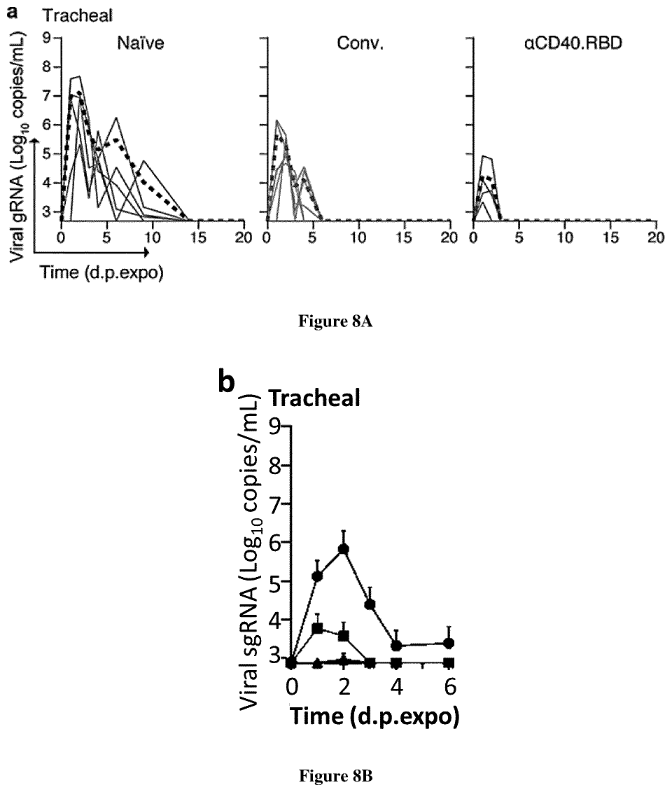

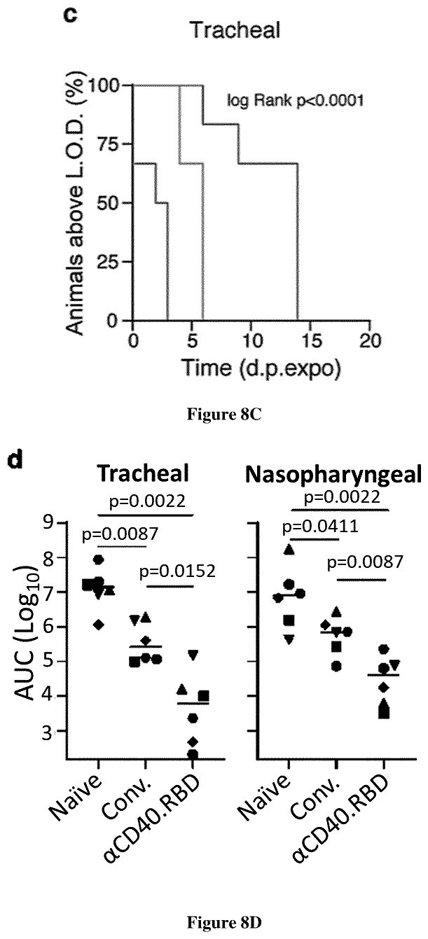

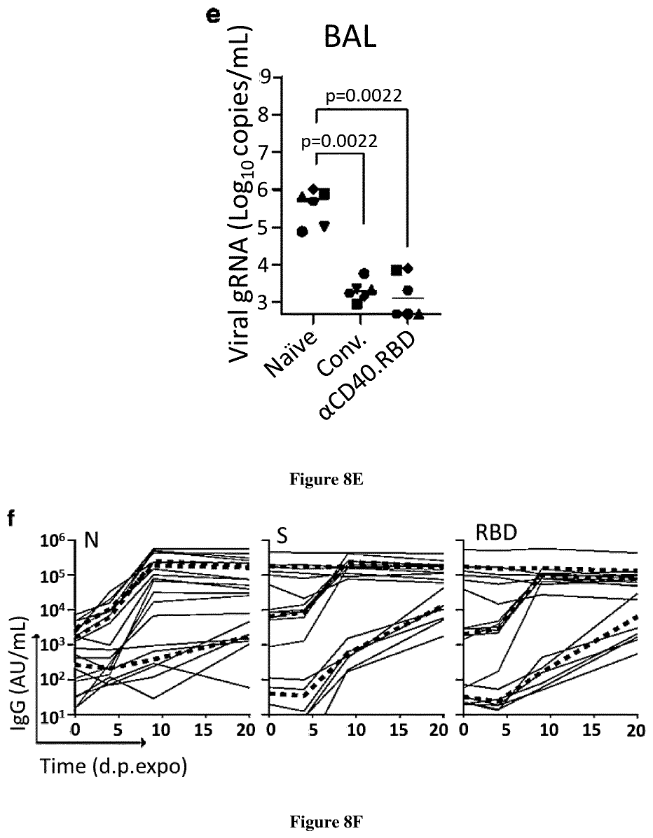

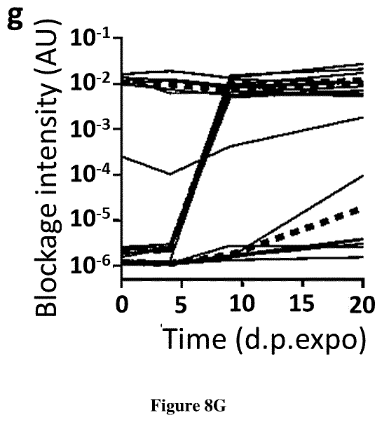

Here, we tested whether such HIS-mice can initiate B- and T-cell immunity in response to CD40 targeting SARS-CoV2 RBD protein adjuvanted with a TLR3/7 agonist (Poly(IC). Poly(IC)/aCD.RBD was administered either alone at weeks 0, 3, and 5 (group 2) or at weeks 3 and 5, following a prime at week 0 with a Drep vaccine encoding the SARS-CoV2 S protein (group 3) (FIG. 1A). The control group 1 consisted of half of the animals injected with PBS or Poly(IC). We monitored the immune responses at day 21 (3 weeks after the priming injection) and at termination (one week after the last booster injection). The reconstitution of human immune system was similar across the groups (FIG. 1B: blood at baseline; FIG. 1C: spleen at sacrifice).

FIG. 2. Induction of circulating Ab-secreting hu-B cells in vaccines. The frequency of antibody-secreting cells (hCD45+ hCD19+ hCD27+ hCD38+ mCD45−) was evaluated by flow cytometry at day 21 in the blood of HIS mice (A) and at sacrifice in the blood (B) and spleen (C).

FIG. 3. The aCD40-RBD+Drep-S priming elicits S-specific IgG+ hu-B cells. The frequency of Spike-specific IgG-switched hu-B cells (hCD45+ hCD19+ hIgG+ Spike+ mCD45−) was evaluated by flow cytometry using the biotinylated SARS-CoV2 spike at day 21 in the blood of HIS mice (A) and at sacrifice in the spleen (B).

FIG. 4. The aCD40-RBD vaccine elicits the expansion of CM CD4+ hu-T cells at 21 d.p.i. and the emergence of EM CD4+ T cells at 42 d.p.i. The frequency of Effector memory hu-CD4+ T cells (hCD45+ hCD3+ hCD4+ hCD27− hCD45RA−mCD45−) and Central memory hu-CD4+ T cells (hCD45+hCD3+hCD4+hCD27+hCD45RA- mCD45−) was evaluated by flow cytometry in the blood of HIS mice at baseline, day 21 and sacrifice.

FIG. 5. The aCD40-RBD vaccine elicits the expansion of CM CD4+ hu-T cells at 21 d.p.i. and the emergence of EM CD4+ T cells at 42 d.p.i. The frequency of Effector memory hu-CD8+ T cells (hCD45+ hCD3+ hCD4− hCD27− hCD45RA− mCD45−) and Central memory hu-CD8+ T cells (hCD45+ hCD3+ hCD4+ hCD27+ hCD45RA − mCD45−) was evaluated by flow cytometry in the blood of HIS mice at baseline, day 21 and sacrifice.

FIG. 6. The aCD40-RBD vaccine elicits the expansion of CM CD4+ hu-T cells at 21 d.p.i. and the emergence of EM CD4+ T cells at 42 d.p.i. The frequency of Stem cell-like memory hu-CD8+ T cells (hCD45+ hCD3+ hCD8+ hTbet+ hCD45RA+ hCD62L+ hCD95+ hCD12230 mCD45−) was evaluated by flow cytometry in the blood (A) and spleen (B) of HIS mice at sacrifice.

FIGS. 7A-7G. SARS-CoV-2 specific B- and T-cell responses induced by aCD40.RBD in convalescent NHP. a. Study design in cynomolgus macaques. b. Relative MFI of IgG binding to SARS-CoV-2 S protein, measured using a Luminex-based serology assay, in serum samples (mean±SD of 6 animals per group). The vertical dotted lines indicate viral exposure and vaccination, respectively. c SARS-CoV-2 S protein-specific binding before any exposure to SARS-CoV-2 (week-26) and on the week of vaccine injection (week 0) in macaques (n=12) compared to convalescent humans (n=7) sampled 24 weeks after the onset of symptoms. The horizontal dotted line represents the background threshold and bars indicate the mean of each group. d. Quantification of SARS-CoV-2 antibodies against RBD measured in the serum of NHPs using a multiplexed solid-phase chemiluminescence assay. Each plain line indicates the individual values, and the bold dotted lines represent the mean for each experimental group. e. Quantification of antibody-induced inhibition of ACE-2 binding in NHP serum. Symbols are as for d. f Frequency of RBD-specific Th1 CD4+ T cells (CD154+ and IFN-γ±IL-2±TNF-α) in the total CD4+ T cell population for each non-immunized convalescent macaque (n=6, blue lines and symbols) and αCD40.RBD-vaccinated convalescent macaque (n=6, green lines and symbols). PBMC were stimulated overnight with SARS-CoV-2 RBD overlapping peptide pools. Time points in each experimental group were compared using the Wilcoxon signed rank test. g. Frequency of cytokine producing cells in the RBD-specific CD4+ T cells (CD154+) for non-immunized convalescent macaque (left) and αCD40.RBD-vaccinated convalescent macaque (right). Each bar indicated the mean of the 6 vaccinated convalescent macaques±SD. Distribution of cytokines is indicated within each bar. BL: Baseline approximately 1 week before immunization; “Post imm.”: Two weeks post immunization.

FIGS. 8A-8G. Efficacy of αCD40.RBD in convalescent cynomolgus macaques.

a. Genomic viral RNA (gRNA) quantification in tracheal swabs of naïve (left, gray lines), convalescent (middle, blue lines), and αCD40.RBD-vaccinated convalescent macaques (right, green lines). The bold line represents the mean viral load for each experimental group. b. Mean of subgenomic (sgRNA) viral loads in tracheal swabs. Data are presented as mean values±SD for each experimental group (n=6 NHP/group). c. Percentage of macaques with viral gRNA above the limit of detection (LOD) over time in tracheal swabs. Experimental groups were compared using log Rank tests; two-tailed p value is indicated. d. Area under the curve (AUC) of gRNA viral loads in tracheal (left panel) and nasopharyngeal swabs (right panel). e. gRNA viral quantification in BAL three days post-exposure (d.p. expo). d, e Each plot represents one macaque (n=6 NHP/group) and bars indicate the mean of each group. Groups were compared using the two-tailed non-parametric Mann-Whitney test. f Quantification of SARS-CoV-2 IgG binding N, S, and RBD after challenge. Each plain line indicates individual values, and the bold dotted lines represent the mean for each experimental group. g. Quantification of antibody-induced inhibition of ACE-2 binding. Lines as in f.

### EXAMPLE 1

Material & Methods

The 20-week-old female NSG (NOD.Cg-Prkdcscid Il2rgtm1Wjl/SzJ) humanized mice (hu-mice) were supplied by the Jackson Laboratories (Bar Harbor, ME, USA) under MTA #1720. Five donors provided hematopoietic stem cells for human immune system reconstitution of the mice. The level of human immune cells reconstitution reached an average of 70%. The hu-mice were housed in Mondor Institute of Biomedical Research infrastructure facilities (U955 INSERM-Paris East Creteil University, Ile-de-France, France) in micro-isolators under pathogen-free conditions with human care, at a temperature of 20-24° C. with 50% +/−15% humidity and a 12-h light/12-h dark cycle. The protocols were approved by the institutional ethical committee “Comité d'Ethique Anses/ENVA/UPEC (CEEA-016)” under statement number 20-043 #25329. The study was authorized by the “Research, Innovation and Education Ministry” under registration number 25329-2020051119073072 v4.

Vaccination of Humanized Mice.

The hu-mice received immunizations at week 0,3, and 5. The priming injection was an intraperitoneal administration of 10 μg of αCD40-RDB adjuvanted with 50 μg of polyinosinic-polycytidylic acid (Poly-IC;Invivogen) combined or not with an intramuscular injection of DREP-S (10 μg). Then hu-mice received booster i.p injections of αCD40-RDB (10 μg) plus Poly-IC (50 μg). Blood was collected at weeks 0 (before immunization), 3, and 6. Hu-mice were euthanized at week 6.

SARS-CoV-2 S Protein-Specific B Cell Analysis

Hu-mice PBMC from 3 weeks after the priming immunization and hu-mice PBMC and spleen cells from 6 weeks (one week after the last recall injection) were incubated first with the biotinylated SARS-CoV-2 S protein for 30 min at 4° C. After a washing step, cells were stained for 30 min at 4° C. with streptavine-AF700 (1:10, ThermoFisher Scientific), anti-human (h) CD45-PeCy7 (1:50, # 2120080, HI30, Sony), anti-mouse (m) CD45-BV711 (1:50, # 1115735, 30F11, Sony), anti-hCXCR4-Pe-Dazzle (1:50, # 12-9999-42, 12G5, eBiosciences), anti-hCCR10-PE (1:50, #314305; R&D System), anti-CD3-BV510 (1:50, #2102240, UCHT1, Sony), anti-CD4-FITC (1:50, #2187040, OKT4, Sony), anti-CD8-PerCpCy5.5 (1:50, #2323550, SKI, Biolegend) antibodies and the following B cell-specific antibodies: anti-hCD19-BV421 (1:16, #2111170, HIB19, Sony), anti-hCD20-APC (1:50, #2111550, 2H7, Sony), anti-hIgG-BV786 (1:16, #564230, G18-145, BD Biosciences), anti-hCD38-APC-Cy7 (1:16, # 2117670, HIT2, Sony). Staining on spleen cells also included a viability marker (LiveDead aqua or yellow stain ThermoFisher Scientific). Cells were washed twice with FACS buffer (PBS 1% FCS) and acquired on the LSRII flow cytometer (BD Biosciences). Analyses were performed on FlowJo v.10.7.1.

Result

The immunogenicity of the aCD40-RBD vaccine (i.e. the antibody comprising the i) heavy chain that is fused to the RBD polypeptide and ii) the light chain as set forth in SEQ ID NO:43) given in homologous or heterologous prime/boost vaccination strategies was studied according to the protocol described in FIG. 1. The results are depicted in FIG. 1-6. In particular, we show that the vaccine induces circulating Ab-secreting hu-B cells (FIG. 2), elicits S-specific IgG+ hu-B cells (FIG. 3), elicits the expansion of central memory CD4+ hu-T cells at 21 d.p.i. and the emergence of effector memory CD4+ T cells at 42 d.p.i (FIG. 4), elicits the expansion of central memory CD8+ hu-T cells at 21 d.p.i. and the emergence of effector memory CD8+ T cells at 42 d.p.i (FIG. 5) and finally induces Stem-cell like memory hu-CD8+ T cells at 42 d.p.i. (FIG. 6). Indeed a single injection of αCD40.RBD (10 μg), adjuvanted with polyinosinic-polycytidylic acid (Poly-IC, 50 μg), by the intraperitoneal route was sufficient to elicit SARS-CoV-2 S protein-specific IgG-switched human B cells in the blood of 50% of immunized mice (FIG. 3). At week 6, one week after the last αCD40.RBD boost, unbiased t-SNE analysis of the splenic human CD19+ B cells revealed cell clusters corresponding to well described subsets of terminally differentiated plasma cells (PCs), early plasma blasts (PBs), and a contingent of PBs and immature PCs in the vaccine groups but not controls (data not shown). At the same time point, splenic SARS-CoV-2 S protein-specific IgG switched human B cells were detected in all vaccinated hu-mice (data not shown), mainly of the PB and immature PC phenotype.

All spike protein-specific IgG-switched human B cells expressed CXCR4 and a discrete cell island was observed in the t-SNE analysis driven by high expression of CCR10 (data not shown), which was confirmed using manual back gating (data not shown). We next evaluated the capacity of the vaccines to induce specific and functional CD4+ and CD8+ memory T cells. The Th1 (IFN-γ+/−IL-2+/− TNF-α) type CD4+ T-cell responses and IFNγ-secreting CD8+ T-cells were observed for the vaccinated hu-mice following ex vivo stimulation of splenocytes with RBD peptide pools (data not shown). We confirmed the presence of human CD8+ T cells specific for the predicted optimal epitopes from SARS-CoV-2 RBD protein in the spleens of vaccinated hu-mice using HLA-I tetramers (data not shown). Subunit vaccines could also be considered as boosters for other type of vaccines in human vaccination campaigns. Thus, in addition to a homologous prime-boost regimen, we tested the capacity of αCD40.RBD to boost heterologous priming with a vector-based vaccine. The DNA-launched self-amplifying RNA replicon vector encoding the SARS-CoV-2 spike glycoprotein (DREP)-S is a previously described platform²⁸ based on the alphavirus genome encoding the genes for the viral RNA replicase but lacking those encoding the structural proteins of the virus^{29.}

We demonstrated that in the two vaccinated groups, the prime boost strategy containing αCD40.RBD efficiently elicited B- and T-cell SARS-CoV-2 specific responses (FIG. 2). In both vaccinated groups, we showed an expansion of effector memory CD4 and CD8+ T cells (CD45RA⁻CD27⁻).

### EXAMPLE 2: The αCD40.RBD Vaccine Recalls Specific Immune Responses in Convalescent Macaques and Improves the Protection of Convalescent Macaques Against SARS-CoV-2 Reinfection

Material & Methods

Cynomolgus macaques (Macaca fascicularis), aged 37-58 months (8 females and 13 males) and originating from Mauritian AAALAC certified breeding centers were used in this study. All animals were housed in IDMIT facilities (CEA, Fontenay-aux-roses), under BSL-3 containment (Animal facility authorization #D92-032-02, Préfecture des Hauts de Seine, France) and in compliance with European Directive 2010/63/EU, the French regulations and the Standards for Human Care and Use of Laboratory Animals, of the Office for Laboratory Animal Welfare (OLAW, assurance number #A5826-01, US). The protocols were approved by the institutional ethical committee “Comité d′Ethique en Expérimentation Animale du Commissariat á l'Energie Atomique et aux Energies Alternatives” (CEtEA #44) under statement number A20-011. The study was authorized by the “Research, Innovation and Education Ministry” under registration number APAFIS#24434-2020030216532863v1.

Non-Human Primate Study Design.

Convalescent cynomolgus macaques previously exposed to SARS-CoV-2 and used to assess hydroxychloroquine (HCQ) and azithromycin (AZTH) antiviral efficacy. None of the AZTH neither HCQ nor the combination of HCQ and AZTH showed a significant effect on viral replication5. Six months (24-26 weeks) post infection (p.i.), twelve of these animals were randomly assigned in two experimental groups. The convalescent vaccinated group (n=6) received 200 μg of αCD40.RBD vaccine by subcutaneous (SC) route diluted in PBS and without any adjuvant. The other six convalescent animals were used as controls and received the equivalent volume of PBS by SC. The two groups of convalescent animals were sampled at week 2 and 4 following vaccine or PBS injection for anti-SARS-CoV-2 immune response evaluation. Additional six age matched (43.7 months±6.76) cynomolgus macaques from same origin were included in the study as controls naïve from any exposure to SARS-CoV-2.

Experimental Infection of Macaques with SARS-CoV-2.

Four weeks after immunization, all animals were exposed to a total dose of 106 pfu of SARS-CoV-2 virus (hCoV-19/France/ 1DF0372/2020 strain; GISAID EpiCoV platform under accession number EPI_ISL_406596) via the combination of intranasal and intratracheal routes (0.25 mL in each nostril and 4.5 mL in the trachea, i.e. a total of 5 mL; day 0), using atropine (0.04 mg/kg) for pre-medication and ketamine (5 mg/kg) with medetomidine (0.05 mg/kg) for anesthesia. Nasopharyngeal, tracheal and rectal swabs, were collected at 1, 2, 3, 4, 6, 9, 14, and days post exposure (d.p.exp.) while blood was taken at 2, 4, 6, 9, 14, and 20 d.p.exp. Bronchoalveolar lavages (BAL) were performed using 50 mL sterile saline at 3 d.p.exp in order to be close to the peak of viral replication and to be able to observe a difference between the vaccinated and control groups. In our earlier study30, we found that at later time-points, viral loads in the BAL were very low or negative. Chest CT was performed at baseline and at 2 and 6 d.p.exp. on anesthetized animals using tiletamine (4 mg/kg) and zolazepam (4 mg/kg). Lesions were scored as we previously described³⁰. Blood cell counts, hemoglobin, and hematocrit were determined from EDTA blood using a DXH800 analyzer (Beckman Coulter).

Evaluation of Anti-Spike, Anti-RBD, and IgG Inhibiting Antibodies.

Anti-Spike IgG from human and NHP sera were titrated by multiplex bead assay. Briefly, Luminex beads were coupled to the Spike protein as previously described6 and added to a Bio-Plex plate (BioRad). Beads were washed with PBS 0.05% tween using a magnetic plate washer (MAG2× program) and incubated for 1 h with serial diluted individual serum. Beads were then washed and anti-NHP IgG-PE secondary antibody (Southern Biotech, clone SB108a) was added at a 1:500 dilution for 45 min at room temperature. After washing, beads were resuspended in a reading buffer 5 min under agitation (800 rpm) on the plate shaker then read directly on a Luminex Bioplex 200 plate reader (Biorad). Average MFI from the baseline samples were used as reference value for the negative control. The amount of anti-Spike IgG was reported as the MFI signal divided by the mean signal for the negative controls. Human sera from convalescent patients who were hospitalized with virologically confirmed COVID-19 were collected three months after symptoms recovery and used as controls for the titration of anti-Spike antibodies.

Anti-RBD and anti-Nucleocapside (N) IgG were titrated using a commercially available multiplexed immunoassay developed by Mesoscale Discovery (MSD, Rockville, MD) as previously described7. Briefly, antigens were spotted at 200-400 μg/mL in a proprietary buffer, washed, dried, and packaged for further use (MSD® Coronavirus Plate 2). Then, plates were blocked with MSD Blocker A following which reference standard, controls and samples diluted 1:500 and 1:5000 in diluent buffer were added. After incubation, detection antibody was added (MSD SULFO-TAG™ Anti-Human IgG Antibody) and then MSD GOLD™ Read Buffer B was added and plates read using a MESO QuickPlex SQ 120MM Reader. Results were expressed as arbitrary unit (AU)/mL.

The MSD pseudo-neutralization assay was used to measure antibodies neutralizing the binding of the spike protein to the ACE2 receptor. Plates were blocked and washed as above, assay calibrator (COVID-19 neutralizing antibody; monoclonal antibody against S protein; 200 μg/mL), control sera, and test sera samples diluted 1:10 and 1:100 in assay diluent were added to the plates. Following incubation of the plates, an 0.25 μg/mL solution of MSD SULFO-TAG™ conjugated ACE-2 was added after which plates were read as above. Electrochemioluminescence (ECL) signal was recorded and results expressed as 1/ECL.

Statistical Analysis.

Data were collected using classical Excel files (Microsoft Excel 2016). Differences between unmatched groups were compared using an unpaired t-test or the Mann-Whitney U test (Graphpad Prism 8.0), and differences between matched groups were compared using a paired t-test or the Wilcoxon signed-rank test (Graphpad Prism 8.0). Viral kinetic parameter was compared using log-rank tests (Graphpad Prism 8.0). Correlation between viral and immune parameter was determined using nonparametric Spearman correlation (Graphpad Prism 8.0).

Result

The immunogenicity observed in the hu-mice model are consistent with those of our previous CD40-targeted influenza and HIV vaccine studies^{21,22,26,27} and demonstrate that αCD40.RBD could be a potent prime or boost vaccine for eliciting RBD-specific T- and B-cell responses19. We thus subcutaneously injected six convalescent cynomolgus macaques with 200 μg of the vaccine without adjuvant. An additional 12 animals (six convalescent and six naive) were injected with PBS as controls (FIG. 7a). All the convalescent macaques, randomly distributed between the vaccine and control groups, had been infected approximately six months before (range=26-24 weeks) with SARS-CoV-2 in a study to evaluate pre-exposure or post-exposure prophylaxis with hydroxychloroquine (HCQ). No evidence of antiviral efficacy³⁰ of HCQ was observed and after this first exposure to the virus, all animals developed similar profiles of viral load (data not shown) and suffered from transient and moderate disease, resulting in increased levels of anti-S IgG antibodies detected in the serum (FIG. 7b). At the time of the αCD40.RBD-vaccine injection, anti-S IgG levels in the two groups of convalescent macaques were comparable and in the average range of specific responses detected in the sera of convalescent patients (FIG. 7c).

Before vaccination, the infection of macaques with SARS-CoV-2 generated both anti-RBD antibodies (FIG. 7d) and low but detectable levels of antibodies inhibiting the binding of the spike protein to the ACE2 receptor (FIG. 7e). Before vaccination, low Th1 (IFN-γ+/−IL-2+/−TNF-α) type CD4+ T-cell responses were observed for both groups of convalescent macaques following ex vivo stimulation of PBMCs with RBD and N-peptide pools (data not shown). None of the convalescent animals had detectable anti-RBD or anti-N CD8+ T cells (data not shown). Two weeks after αCD40.RBD vaccine injection, all six vaccinated macaques exhibited significantly increased levels of anti-S (FIG. 7b) and anti-RBD IgG (FIG. 7d) in the serum, which correlated with an increased capacity of inhibition of RBD binding to the ACE2 receptor (p=0.022, FIG. 7e), as they remained elevated four weeks after vaccination. In an in vitro assay using authentic viruses¹⁴, we confirmed that antibodies raised by the vaccine not only neutralizes the variant containing the D614G present in the αCD40.RBD (data not shown) but also cross neutralizes B1.1.7 and to a lesser extent B1.351 known to be partially resistant to antibodies raised by previously circulating variants^{31,32}. None of these parameters increased in PBS-injected convalescent controls (FIG. 7b,d,e). In addition, anti-S IgG levels in the vaccinated macaques were higher (p=0.0018) than those typically observed in humans 1 to 3 months after symptomatic SARS-CoV-2 infection (data not shown). The immunization also elicited a significant increase in the anti-RBD Th1 response in all six immunized animals (p=0.031; FIG. 7f,g), whereas no changes in the magnitude of anti-N CD4+ T cells (data not shown) or SARS-CoV-2 specific CD8+ T cells was observed (data not shown).

Four weeks following vaccine or placebo injection, the 12 convalescent macaques were exposed a second time to a high dose (1×106 pfu) of SARS-CoV-2 administered via the combined intra-nasal and intra-tracheal route using a previously reported challenge procedure³⁰. Six SARS-CoV-2 naive animals were also challenged as controls. All naive animals became infected, as shown by the detection of viral genomic (gRNA) and sub-genomic (sgRNA) RNA in tracheal (FIG. 8a-d) and nasopharyngeal (FIG. 8d) swabs and broncho-alveolar lavages (BAL, FIG. 8e). Of note, the dynamics of viral replication in these animals was comparable to that observed during the first infection six months earlier in the two groups of convalescent macaques (data not shown).

The non-vaccinated convalescent animals were not protected against the second SARS-CoV-2 challenge, but significantly lower viral RNA levels were detected in the upper respiratory tract than in the naive animals (FIG. 8a-e). The αCD40.RBD vaccine remarkably improved the partial protection observed in the convalescent macaques. All vaccinated animals exhibited significantly lower viral gRNA levels (p=0.015, FIG. 8d) than the non-vaccinated convalescent animals. The levels of sgRNA remained below the limit of detection in upper respiratory tract samples for 5 of 6 vaccinated animals, whereas sgRNA was detected in 4 of 6 non-vaccinated convalescent and all naïve control animals (data not shown). Moreover, the time post-exposure (p.expo.) to reach undetectable gRNA levels was significantly lower in vaccinated convalescent than nonvaccinated and control animals (FIG. 8c). The efficacy of vaccination was also higher in the lower respiratory tract, as only 3 of 6 vaccinated macaques were above the limit of detection for gRNA in BAL at day 3 p.expo. versus day 6 for the six non-vaccinated convalescent animals (FIG. 8e). Complete protection from shedding of the virus from the gastrointestinal tract was noted in the non-vaccinated and vaccinated convalescent macaques (data not shown), indicating that in addition to vaccine, the natural infection immunity could play an important role to prevent secondary viral transmission^{33.}

The reduction of viral load in vaccinated and non-vaccinated convalescent macaques relative to naive infected animals was associated with a limited impact on leukocyte numbers (data not shown) and reduced cytokine concentrations in the plasma, in particular those of IL-1RA and CCL2 (data not shown). Such viral loads and cytokine profiles were also associated with a reduction in lung lesions (data not shown), as scored by X-ray computerized tomography (CT). We then analyzed the immune responses of all animals following SARS-CoV-2 viral challenge. The naive controls showed the slowest development of anti-S, anti-RBD, and anti-N IgG (FIG. 8f), of which the levels remained significantly lower than for the other two groups at day 20 p.expo. (p=0.022). The non-vaccinated convalescent animals raised a rapid and robust anamnestic antibody response (FIG. 8f), which was associated with a significant increase (p=0.031) in the serum capacity to neutralize ACE2 binding to RBD (FIG. 8g) by p.expo. day 9, reaching at that time the levels observed in the vaccinated group. The anti-S- and anti-RBD-specific antibody responses and neutralization activity of the serum was maintained in the vaccinated macaques at the high levels already achieved at the time of challenge and remained superior to that of the control macaques (FIG. 8f, g). The anti-RBD Th1 CD4+ response increased post-challenge for most of the control (convalescent and naive) animals, with higher levels for some of the naïve controls as early as p.expo. day 9 (data not shown). On the contrary, all 18 animals showed comparable antibody and CD4+ T cell responses to the N-peptide pool (data not shown), probably reflecting a predominance of the response against nonstructural antigens in infected individuals. The IFN-γ-mediated CD8+ T-cell response was also mainly directed against the N peptides (data not shown), but with a significantly reduced intensity in all convalescent macaques than in the naive controls (data not shown), probably reflecting the lower exposure to viral antigens as a result of better control of viral replication. Spearman analysis between all recorded parameters revealed that the induction of anti-RBD- and ACE2-inhibiting antibodies was the strongest parameter to correlate with the reduction of viral load and disease markers, as were the plasma levels of the inflammatory cytokines IL-1RA and CCL2 (data not shown).

Discussion

In humans, the durability of protection induced by natural SARS-CoV-2 infection and the first vaccine candidates is unknown. In convalescent humans, the virus neutralizing-antibody response wanes and re-infections have been reported within months following previous exposure^{33,34}. The decrease in neutralizing antibody levels observed in most patients within three months post-infection may suggest that vaccine boosters will be required to provide long-lasting protection³⁵. In contrast to previous NHP re-challenge studies performed shortly after a first infection³⁶, we demonstrate that SARS-CoV-2 reinfection is not fully prevented in convalescent macaques six months after initial exposure to the virus, confirming that protective immunity wanes over time. In addition, the vaccines currently used in humans are aimed at preventing severe disease and only partial information is available as to their capacity to prevent infection and reduce initial viral replication to the level required to significantly limit secondary transmission. Vaccinated individuals who develop an asymptomatic or mild symptomatic infection may continue transmitting the virus and actively contribute to circulation of the virus. The αCD40.RBD vaccine we developed significantly improved immunity of convalescent macaques, resulting in a reduction of viral load following re-exposure to the virus down to levels that may avoid such secondary transmission. This vaccine may therefore represent an appropriate booster of pre-existing immunity, either induced by natural infection or previous priming with vector-based vaccines. This new-generation subunit vaccine targeting the antigen to CD40-expressing cells, may have advantages for a safe and efficient boosting strategy. The capacity to induce protective immunity without requiring an adjuvant would accelerate the development of a protein-based vaccine with expected improved tolerability over adjuvanted vaccines and thus suitable for people with specific vulnerabilities and children, an important part of the population to consider in the control of circulation of the virus.

References:

Throughout this application, various references describe the state of the art to which this invention pertains. The disclosures of these references are hereby incorporated by reference into the present disclosure.
1. Voysey, M. et al. Safety and efficacy of the ChAdOx1 nCoV-19 vaccine (AZD1222) against SARS-CoV-2: an interim analysis of four randomized controlled trials in Brazil, South Africa, and the UK. Lancet (London, England) 397,99-111, (2020).
2. Corbett, K. S. et al. Evaluation of the mRNA-1273 vaccine against SARS-CoV-2 in nonhuman primates. N. Engl. J. Med 383,1544-1555 (2020).
3. Polack, F. P. et al. Safety and efficacy of the BNT162b2 mRNA Covid-19 vaccine. N. Engl. J. Med. 383,2603-2615 (2020).
4. Mercado, N. B. et al. Single-shot Ad26 vaccine protects against SARS-CoV-2 in rhesus macaques. Nature 586,583-588 (2020).
5. Oliver, S. E. et al. The Advisory Committee on Immunization Practices' Interim Recommendation for Use of Pfizer-BioNTech COVID-19 Vaccine—United States, December 2020. MMWR Morb. Mortal. Wkly Rep. 69,1922-1924 (2020).
6. Widge, A. T. et al. Durability of responses after SARS-CoV-2 mRNA-1273 Vaccination. N. Engl. J. Med. 384, 80-82 (2020).
7. Anderson, E. J. et al. Safety and immunogenicity of SARS-CoV-2 mRNA-1273 vaccine in older adults. N. Engl. J. Med. 383,2427-2438 (2020).
8. Ella, R. et al. Safety and immunogenicity of an inactivated SARS-CoV-2 vaccine, BBV152: interim results from a double-blind, randomised, multicentre, phase 2 trial, and 3-month follow-up of a double-blind, randomised phase 1 trial. Lancet Infect Dis. 7,950-961 (2021).
9. Levine-Tiefenbrun, M. et al. Initial report of decreased SARS-CoV-2 viral load after inoculation with the BNT162b2 vaccine. Nat. Med. 27,790-782 (2021).
10. Logunov, D. Y. et al. Safety and efficacy of an rAd26 and rAd5 vector-based heterologous prime-boost COVID-19 vaccine: an interim analysis of a randomised controlled phase 3 trial in Russia. Lancet (Lond., Engl.) 397,
   71-681 (2021).
11. Wu, Z. et al. Safety, tolerability, and immunogenicity of an inactivated SARSCoV-2 vaccine (CoronaVac) in healthy adults aged 60 years and older: a randomised, double-blind, placebo-controlled, phase ½ clinical trial. Lancet Infect. Dis. 21,803-812 (2021).
12. Ebinger, J. E. et al. Antibody responses to the BNT162b2 mRNA vaccine in individuals previously infected with SARS-CoV-2. Nat. Med. 27, 981-984 (2021).
13. Emary, K. R. W. et al. Efficacy of ChAdOx1 nCoV-19 (AZD1222) vaccine against SARS-CoV-2 variant of concern 202012/01 (B.1.1.7): an exploratory analysis of a randomised controlled trial. Lancet 397, 1351-1362 (2021).
14. Planas, D. et al. Sensitivity of infectious SARS-CoV-2 B.1.1.7 and B.1.351 variants to neutralizing antibodies. Nature medicine 27, 917-924 (2021).
15. Sapkal, G. N. et al. Inactivated COVID-19 vaccine BBV152/COVAXIN effectively neutralizes recently emerged B 1.1.7 variant of SARS-CoV-2. J Travel Med. 28, 051 (2021).
16. Schiller, J. T. & Lowy, D. R. Raising expectations for subunit vaccine. J. Infect. Dis. 211, 1373-1375 (2015).
17. Ma, X. et al. Nanoparticle vaccines based on the receptor binding domain (RBD) and heptad repeat (HR) of SARS-CoV-2 elicit robust protective immune responses. Immunity 53, 1315-1330.e1319 (2020).
18. Brouwer, P. J. M. et al. Two-component spike nanoparticle vaccine protects macaques from SARS-CoV-2 infection. Cell 184, 1188-1200 (2021).
19. McMahan, K. et al. Correlates of protection against SARS-CoV-2 in rhesus macaques. Nature 590, 630-634 (2021).
20. Yin, W. et al. Functional specialty of CD40 and dendritic cell surface lectins for exogenous antigen presentation to CD8(+) and CD4(+) T cells. EBioMedicine 5, 46-58 (2016).
21. Godot, V. et al. TLR-9 agonist and CD40-targeting vaccination induces HIV-1 envelope-specific B cells with a diversified immunoglobulin repertoire in humanized mice. PLoS Pathog. 16, e1009025 (2020).
22. Cheng, L. et al. TLR3 agonist and CD40-targeting vaccination induces immune responses and reduces HIV-1 reservoirs. J. Clin. Invest. 128, 4387-4396 (2018).
23. Bouteau, A. et al. DC subsets regulate humoral immune responses by supporting the differentiation of distinct Tfh cells. Front. Immunol. 10, 1134 (2019).
24. Zurawski, G. et al. Superiority in Rhesus Macaques of targeting HIV-1 Env gp140 to CD40 versus LOX-1 in combination with replication-competent NYVAC-KC for induction of Env-specific antibody and T cell responses. J. Virol. 91, 01596-01616 (2017).
25. Shang, J. et al. Cell entry mechanisms of SARS-CoV-2. Proc. Natl Acad. Sci. USA 117, 11727-11734 (2020).

1.-43. canceled.

44. An antibody that is directed against a surface antigen of an antigen presenting cell wherein the heavy chain and/or the light chain is conjugated or fused to a RBD polypeptide having at least 90% of identity with the amino acid sequence that ranges from the amino acid residue at position 319 to the amino acid residue at position 541 in SEQ ID NO:1.

45. The antibody of claim 44, wherein the RBD polypeptide consists of:
a) the amino acid that ranges from the amino acid residue at position 319 to the amino acid residue at position 541 in SEQ ID NO:1;
b) the amino acid that ranges from the amino acid residue at position 319 to the amino acid residue at position 541 in SEQ ID NO:1 and that comprises the C538S non-naturally occurring mutation;
c) the amino acid that ranges from the amino acid residue at position 319 to the amino acid residue at position 541 in SEQ ID NO:1 and that comprises the C538S non-naturally occurring mutation and one or more naturally occurring mutation(s) selected from the group consisting of K417N, K417T, E484K, and N501Y mutations;
d) the amino acid that ranges from the amino acid residue at position 319 to the amino acid residue at position 541 in SEQ ID NO:1 and that comprises the K417T, E484K, N501Y naturally-occurring mutations and the non-naturally occurring mutation C538S mutation;
e) the amino acid that ranges from the amino acid residue at position 319 to the amino acid residue at position 541 in SEQ ID NO:1 and that comprises the K417N, E484K, N501Y naturally occurring mutation and the C538S non-naturally occurring mutation;
f) the amino acid that ranges from the amino acid residue at position 319 to the amino acid residue at position 541 in SEQ ID NO:1 and that comprises the K417N, E484Q, N501Y naturally-occurring mutations and the non-naturally occurring mutation C538S mutation;
g) the amino acid that ranges from the amino acid residue at position 319 to the amino acid residue at position 541 in SEQ ID NO:1 and that comprises the K417T, E484Q, N501Y naturally-occurring mutations and the non-naturally occurring mutation C538S mutation.
h) the amino acid that ranges from the amino acid residue at position 319 to the amino acid residue at position 541 in SEQ ID NO:1 and that comprises the K417N, L452R, T478K, E484K, N501Y naturally-occurring mutations and the non- naturally occurring mutation C538S mutation; or
i) the amino acid that ranges from the amino acid residue at position 319 to the amino acid residue at position 541 in SEQ ID NO:1 and that comprises the K417N, L452R, T478K, E484Q, N501Y naturally occurring mutation and the C538S non-naturally occurring mutation.

46. The antibody of claim 44, wherein:
a) the heavy chain of the antibody is conjugated or fused to the RBD polypeptide;
b) the light chain of the antibody is conjugated or fused to the RBD polypeptide; or
c) both the heavy and light chains of the antibody are conjugated or fused to the RBD polypeptide.

47. The antibody of claim 44, wherein:
a) the heavy chain is fused or conjugated to the RBD polypeptide that consists of the amino acid that ranges from the amino acid residue at position 319 to the amino acid residue at position 541 in SEQ ID NO:1 and that comprises the C538S mutation and the light chain is conjugated or fused to the RBD polypeptide consists of the amino acid that ranges from the amino acid residue at position 319 to the amino acid residue at position 541 in SEQ ID NO:1 and that comprises the K417N, E484K, N501Y and C538S mutations; or
b) the light chain is fused or conjugated to the RBD polypeptide that consists of the amino acid that ranges from the amino acid residue at position 319 to the amino acid residue at position 541 in SEQ ID NO:1 and that comprises the C538S mutation and the heavy chain is conjugated or fused to the RBD polypeptide consists of the amino acid that ranges from the amino acid residue at position 319 to the amino acid residue at position 541 in SEQ ID NO:1 and that comprises the K417N, E484K, N501Y and C538S mutations.

48. The antibody of claim 44, which is an IgG antibody.

49. The antibody of claim 44, which is a chimeric antibody or a humanized antibody.

50. The antibody of claim 44, which is specific for CD40.

51. The antibody of claim 50, wherein the antibody:
a) derives from
the 12E12 antibody and comprises:
a heavy chain comprising the complementarity determining regions CDR1H, CDR2H, and CDR3H, the CDR1H having the amino acid sequence GFTFSDYYMY (SEQ ID NO:2), the CDR2H having the amino acid sequence YINSGGGSTYYPDTVKG (SEQ ID NO:3), and the CDR3H having the amino acid sequence RGLPFHAMDY (SEQ ID NO:4),
and a light chain comprising the complementarity determining regions CDR1L, CDR2L, and CDR3L, the CDR1L having the amino acid sequence SASQGISNYLN (SEQ ID NO:5) the CDR2L having the amino acid sequence YTSILHS (SEQ ID NO:6) and the CDR3L having the amino acid sequence QQFNKLPPT (SEQ ID NO:7);
the 11B6 antibody and comprises:
a heavy chain comprising the complementarity determining regions CDR1H, CDR2H, and CDR3H, the CDR1H having the amino acid sequence GYSFTGYYMH (SEQ ID NO:8), the CDR2H having the amino acid sequence RINPYNGATSYNQNFKD (SEQ ID NO:9), and the CDR3H having the amino acid sequence EDYVY (SEQ ID NO:10), and
a light chain comprising the complementarity determining regions CDR1L, CDR2L, and CDR3L, the CDR1L having the amino acid sequence RSSQSLVHSNGNTYLH (SEQ ID NO:11) the CDR2L having the amino acid sequence KVSNRFS (SEQ ID NO:12) and the CDR3L having the amino acid sequence SQSTHVPWT (SEQ ID NO:13); or
the 12B4 antibody and comprises:
a heavy chain comprising the complementarity determining regions CDR1H, CDR2H, and CDR3H, the CDR1H having the amino acid sequence GYTFTDYVLH (SEQ ID NO:14), the CDR2H having the amino acid sequence YINPYNDGTKYNEKFKG (SEQ ID NO:15), and the CDR3H having the amino acid sequence GYPAYSGYAMDY (SEQ ID NO:16), and
a light chain comprising the complementarity determining regions CDR1L, CDR2L, and CDR3L, the CDR1L having the amino acid sequence RASQDISNYLN (SEQ ID NO:17) the CDR2L having the amino acid sequence YTSRLHS (SEQ ID NO:18) and the CDR3L having the amino acid sequence HHGNTLPWT (SEQ ID NO:19); or
b) is selected from:
an antibody mAb1 comprising a heavy chain variable region of sequence SEQ ID NO:20 and a light chain variable region of sequence SEQ ID NO:21;
an antibody mAb2 comprising a heavy chain variable region of sequence SEQ ID NO:22 and a light chain variable region of sequence SEQ ID NO:21;
an antibody mAb3 comprising a heavy chain variable region of sequence SEQ ID NO:23 and a light chain variable region of sequence SEQ ID NO:24;
an antibody mAb4 comprising a heavy chain variable region of sequence SEQ ID NO:25 and a light chain variable region of sequence SEQ ID NO:26;
an antibody mAb5 comprising a heavy chain variable region of sequence SEQ ID NO:27 and a light chain variable region of sequence SEQ ID NO:28; and
an antibody mAb6 comprising a heavy chain variable region of sequence SEQ ID NO:29 and a light chain variable region of sequence SEQ ID NO:30.

52. The antibody of claim 50, which is a CD40 agonist antibody.

53. The antibody of claim 52, wherein the heavy chain or the light chain of the CD40 agonist antibody that is not conjugated or fused to the RBD polypeptides is conjugated or fused to a CD40 binding domain of CD40L (SEQ ID NO:47).

54. The antibody of claim 53, wherein the CD40 binding domain of CD40L is fused to the C-terminus of the light or heavy chain that is not conjugated or fused to the RBD polypeptides of said CD40 agonist antibody, directly or via a linker.

55. The antibody of claim 53, wherein the heavy chain of the antibody is fused or conjugated to the RBD polypeptide and the light chain is conjugated or fused to the CD40 binding domain of CD40L (SEQ ID NO:47).

56. The antibody of claim 44, which is specific for Langerin.

57. The antibody of claim 44, wherein the heavy chain and/or the light chain is fused to the RBD polypeptide via a linker selected from the group consisting of FlexV1 of amino acid sequence SEQ ID NO:37, f1 of amino acid sequence SEQ ID NO:38, f2 of amino acid sequence SEQ ID NO:39, f3 of amino acid sequence SEQ ID NO:40, and f4 of amino acid sequence SEQ ID NO:41.

58. The antibody of claim 44, wherein said antibody:
a) comprises i) a heavy chain that is fused to the RBD polypeptide to form the fusion protein as set forth in SEQ ID NO:42 and ii) a light chain as set forth in SEQ ID NO:43;
b) comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:44;
c) comprises i) a heavy chain that is fused to the RBD polypeptide to form the fusion protein as set forth in SEQ ID NO:45 and ii) a light chain that is fused to the RBD polypeptide to form the fusion protein as set forth in SEQ ID NO:46;
d) comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:48;
e) comprises i) a heavy chain that is fused to the RBD polypeptide to form the fusion protein as set forth in SEQ ID NO:49 and ii) a light chain that is fused to the RBD polypeptide to form the fusion protein as set forth in SEQ ID NO:46;
f) comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:50;
g) comprises i) a heavy chain that is fused to the RBD polypeptide to form the fusion protein as set forth in SEQ ID NO:51 and ii) a light chain that is fused to the RBD polypeptide to form the fusion protein as set forth in SEQ ID NO:46;
h) comprises i) a heavy chain that is fused to the RBD polypeptide to form the fusion protein as set forth in SEQ ID NO:42 and ii) a light chain that is fused to the RBD polypeptide to form the fusion protein as set forth in SEQ ID NO:52; or
i) comprises i) a heavy chain that is fused to the RBD polypeptide to form the fusion protein as set forth in SEQ ID NO:53 and ii) a light chain that is fused to the RBD polypeptide to form the fusion protein as set forth in SEQ ID NO:46.

59. A nucleic acid that encodes for the heavy chain and/or the light chain of the antibody of claim 44.

60. A vector comprising the nucleic acid of claim 59.

61. A host cell which has been transfected, infected, or transformed by the nucleic acid of claim 59 and/or a vector comprising the nucleic acid of claim 59.

62. A vaccine composition comprising the antibody of claim 44.

63. A method for vaccinating a subject in need thereof against SARS-Cov 2 comprising administering a therapeutically effective amount of the antibody of claim 44.


SEQUENCE LISTING (53 sequences)

SEQ ID NO: 1 - protein, 1273 aa, SARS-CoV2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 10 aa, Artificial
  H-CDR1
GFTFSDYYMY

SEQ ID NO: 3 - protein, 17 aa, Artificial
  H-CDR2
YINSGGGSTYYPDTVKG

SEQ ID NO: 4 - protein, 10 aa, Artificial
  H-CDR3
RGLPFHAMDY

SEQ ID NO: 5 - protein, 11 aa, Artificial
  L-CDR1
SASQGISNYLN

SEQ ID NO: 6 - protein, 7 aa, Artificial
  L-CDR2
YTSILHS

SEQ ID NO: 7 - protein, 9 aa, Artificial
  L-CDR3
QQFNKLPPT

SEQ ID NO: 8 - protein, 10 aa, Artificial
  H-CDR1
GYSFTGYYMH

SEQ ID NO: 9 - protein, 17 aa, Artificial
  H-CDR2
RINPYNGATSYNQNFKD

SEQ ID NO: 10 - protein, 5 aa, Artificial
  H-CDR3
EDYVY

SEQ ID NO: 11 - protein, 16 aa, Artificial
  L-CDR1
RSSQSLVHSNGNTYLH

SEQ ID NO: 12 - protein, 6 aa, Artificial
  L-CDR2
KVSNRF

SEQ ID NO: 13 - protein, 9 aa, Artificial
  L-CDR3
SQSTHVPWT

SEQ ID NO: 14 - protein, 10 aa, Artificial
  H-CDR1
GYTFTDYVLH

SEQ ID NO: 15 - protein, 17 aa, Artificial
  H-CDR2
YINPYNDGTKYNEKFKG

SEQ ID NO: 16 - protein, 12 aa, Artificial
  H-CDR3
GYPAYSGYAMDY

SEQ ID NO: 17 - protein, 11 aa, Artificial
  L-CDR1
RASQDISNYLN

SEQ ID NO: 18 - protein, 7 aa, Artificial
  L-CDR2
YTSRLHS

SEQ ID NO: 19 - protein, 9 aa, Artificial
  L-CDR3
HHGNTLPWT

SEQ ID NO: 20 - protein, 116 aa, Artificial
  VH domain
EVQLVQSGAEVKKPGASVKISCKASGYSFTGYYMHWVKQAHGQGLEWIGRINPYNGATSYNQNFKDRATLTVDKSTSTAYMELSSLRSEDTAVYYCAREDYVYWGQGTTVTVSSAS

SEQ ID NO: 21 - protein, 108 aa, Artificial
  VL domain
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTK

SEQ ID NO: 22 - protein, 116 aa, Artificial
  VH domain
EVQLVQSGAEVKKPGASVKVSCKASGYSFTGYYMHWVRQAPGQGLEWIGRINPYNGATSYNQNFKDRVTLTVDKSTSTAYMELSSLRSEDTAVYYCAREDYVYWGQGTTVTVSSAS

SEQ ID NO: 23 - protein, 123 aa, Artificial
  VH domain
EVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAS

SEQ ID NO: 24 - protein, 103 aa, Artificial
  VL domain
DIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTK

SEQ ID NO: 25 - protein, 121 aa, Artificial
  VH domain
DVQLQESGPDLVKPSQSLSLTCTVTGYSITSDYSWHWIRQFPGNKLEWMGYIYYSGSTNYNPSLKSRISITRDTSKNQFFLQLNSVTTEDSATYFCARFYYGYSFFDYWGQGTTLTVSSAS

SEQ ID NO: 26 - protein, 102 aa, Artificial
  VL domain
QIVLTQSPAFMSASPGEKVTMTCSASSSVSYMHWYQQKSGTSPKRWIYDTSKLASGVPARFSGSGSGTSYSLTISSMEAEDAATYYCQQWSSNPLTFGAGTK

SEQ ID NO: 27 - protein, 128 aa, Artificial
  VH domain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSAS

SEQ ID NO: 28 - protein, 103 aa, Artificial
  VL domain
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTK

SEQ ID NO: 29 - protein, 121 aa, Artificial
  VH domain
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSAS

SEQ ID NO: 30 - protein, 103 aa, Artificial
  VL domain
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTK

SEQ ID NO: 31 - protein, 81 aa, Artificial
  VH domain
SVKMSCKASGYTFTDYVISWVKQRTGQGLEWIGDIYPGSGYSFYNENFKGKATLTADKSSTTAYMQLSSLTSEDSAVYFCA

SEQ ID NO: 32 - protein, 76 aa, Artificial
  VL domain
ASISCRSSQSLVHSNGNTYLHWYLQKPGQSPKLLIYKVSNRFSGVPDRFSGSGSGTNFTLKISRVEAEDLGLYFCS

SEQ ID NO: 33 - protein, 82 aa, Artificial
  VH domain
SSVKMSCKASGYTFTDYVISWVKQRTGQGLEWIGDIYPGSGYSFYNENFKGKATLTADKSSTTAYMQLSSLTSEDSAVYFCA

SEQ ID NO: 34 - protein, 74 aa, Artificial
  VL domain
VTLTCRSSTGAVTTSNYANWVQEKPDHLFTGLIGGTNNRVSGVPARFSGSLIGDKAALTITGAQTEDEAIYFCA

SEQ ID NO: 35 - protein, 456 aa, Artificial
  heavy chain
QVQLQQSGAELVRPGASVTLSCKASGYTFIDHDMHWVQQTPVYGLEWIGAIDPETGDTGYNQKFKGKAILTADKSSRTAYMELRSLTSEDSAVYYCTIPFYYSNYSPFAYWGQGALVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCADLLGGPSVFIFPPKIKDVLMISLSPMVTCVVVDVSEDDPDAQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKAS

SEQ ID NO: 36 - protein, 213 aa, Artificial
  light chain
QIVLSQSPAILSASPGEKVTMTCRASSSVSYMHWYQRKPGSSPKPWIYATSNLASGVPARFSGSGSGTSYSLTISRVEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - protein, 27 aa, Artificial
  linker
QTPTNTISVTPTNNSTPTNNSNPKPNP

SEQ ID NO: 38 - protein, 25 aa, Artificial
  Linker
SSVSPTTSVHPTPTSVPPTPTKSSP

SEQ ID NO: 39 - protein, 25 aa, Artificial
  linker
PTSTPADSSTITPTATPTATPTIKG

SEQ ID NO: 40 - protein, 25 aa, Artificial
  linker
TVTPTATATPSAIVTTITPTATTKP

SEQ ID NO: 41 - protein, 25 aa, Artificial
  linker
TNGSITVAATAPTVTPTVNATPSAA

SEQ ID NO: 42 - protein, 671 aa, Artificial
  hAnti-CD40VH3-LV-hIgG4H-C-AS-ViralSARS-CoV-2- Spike-RBD-EPEA
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 43 - protein, 214 aa, Artificial
  hAnti-CD40VK2-LV-hIgGK-C
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 439 aa, Artificial
  hAnti-CD40VK2-LV-hIgGK-C-ViralSARS-CoV-2-Spike- RBDC221S SA var
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNF

SEQ ID NO: 45 - protein, 671 aa, Artificial
  hAnti-CD40VH3-LV-hIgGK-C-ViralSARS-CoV-2-Spike- RBDC221S SA var
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNF

SEQ ID NO: 46 - protein, 439 aa, Artificial
  hAnti-CD40VK2-LV-hIgG4H-C-ViralSARS-CoV-2- Spike-RBDC221S
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 47 - protein, 149 aa, Artificial
  CD40L binding domain
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 48 - protein, 439 aa, Artificial
  hAnti-CD40VK2-LV-hIgGK-C-ViralSARS-CoV-2-Spike- RBDC221S (K417N,L452R, T478K, E484Q, N501Y)
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVQGFNCYFPLQSYGFQPTYGVGYQPYRVVVLSFELLHAPATVCGPKKSTNLVKNKSVNF

SEQ ID NO: 49 - protein, 671 aa, Artificial
  hAnti-CD40VH3-LV-hIgGK-C-ViralSARS-CoV-2-Spike- RBDC221S (K417N,L452R, T478K, E484Q, N501Y)
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVQGFNCYFPLQSYGFQPTYGVGYQPYRVVVLSFELLHAPATVCGPKKSTNLVKNKSVNF

SEQ ID NO: 50 - protein, 439 aa, Artificial
  hAnti-CD40VK2-LV-hIgGK-C-ViralSARS-CoV-2-Spike- RBDC221S (K417N,L452R, T478K, E484K, N501Y)
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKSVNF

SEQ ID NO: 51 - protein, 671 aa, Artificial
  hAnti-CD40VH3-LV-hIgGK-C-ViralSARS-CoV-2-Spike- RBDC221S (K417N,L452R, T478K, E484K, N501Y)
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKSVNF

SEQ ID NO: 52 - protein, 439 aa, Artificial
  hAnti-CD40VK2-LV-hIgGK-C-ViralSARS-CoV-2-Spike- RBDC221S (K417N, E484Q, N501Y)
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVQGFNCYFPLQSYGFQPTYGVGYQPYRVVVLSFELLHAPATVCGPKKSTNLVKNKSVNF

SEQ ID NO: 53 - protein, 671 aa, Artificial
  hAnti-CD40VH3-LV-hIgGK-C-ViralSARS-CoV-2-Spike- RBDC221S (K417N, E484Q, N501Y)
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVQGFNCYFPLQSYGFQPTYGVGYQPYRVVVLSFELLHAPATVCGPKKSTNLVKNKSVNF